(19) United States
(12) Patent Application Publication
(10) Pub. No.: US 2015/0045671 A1
(43) Pub. Date: Feb. 12, 2015

(54) ULTRASOUND PROBE

(71) Applicant: Konica Minolta, Inc., Tokyo (JP)

(72) Inventor: Masashi OZAWA, Kanagawa (JP)

(21) Appl. No.: 14/446,839

(22) Filed: Jul. 30, 2014

(30) Foreign Application Priority Data
Aug. 7, 2013 (JP) 2013-164537

(51) Int. Cl. A61B 8/00

(52) U.S. Cl. CPC A61B 8/4483; A61B 8/4444
USPC 600/459

(57) ABSTRACT

An ultrasound probe includes a laminated body having: a piezoelectric body having a thickness in a first direction; a first electrode and a second electrode that face each other so as to sandwich the piezoelectric body in the first direction; an intermediate layer that is electrically connected with the second electrode and that is provided on an opposite side to the piezoelectric body with respect to the second electrode; and a third electrode that extends in a second direction that is orthogonal to the first direction. A plurality of the first electrodes and second electrodes are arranged at predetermined intervals in the second direction. A plurality of the laminated bodies are arranged in a third direction. A first groove that penetrates through the first electrode, the piezoelectric body, and the second electrode and a part of the intermediate layer is formed in the laminated body.

### BACKGROUND OF THE INVENTION

1. Field of the Invention

The present invention relates to an ultrasound probe for an ultrasound diagnostic imaging apparatus that directs ultrasound into a living body and images tissue information inside the body by means of ultrasound reflected by tissues in the body.

2. Description of the Related Art

Ultrasound diagnostic imaging is a diagnostic imaging method wherein ultrasound is directed into a living body and tissue information inside the body is imaged by means of the ultrasound reflected by biological tissues. A piezoelectric body inside an ultrasound probe generates ultrasound in response to an applied electrical signal, receives ultrasound reflected by tissues in the body, and converts the ultrasound into electrical signals.

FIG. 39 is a perspective view that illustrates electrical interconnection of piezoelectric bodies in a 1.5-D ultrasound transducer array described in U.S. Pat. No. 5,617,865. In the ultrasound transducer illustrated in FIG. 39, electronic scanning can be performed on respective piezoelectric bodies that are divided in the array direction that is indicated by the X-axis, and with grooves 108 and 110 formed therein, aperture control can be performed in the short-axis direction that is indicated by the Y-axis.

In some cases sub-dicing is performed on the above described piezoelectric body in the width direction to prevent interference caused by vibrations in the width direction that is the array direction of the piezoelectric elements. The term “sub-dicing is performed” refers to dividing a piezoelectric element by providing grooves that penetrate through a part or entire of the piezoelectric element. Generally, a piezoelectric body inside an ultrasound probe utilizes a thickness longitudinal vibration that corresponds to a thickness T thereof. However, if a width W of the piezoelectric body is equal to or greater than a predetermined value with respect to the thickness T, in some cases a thickness longitudinal vibration and a width vibration that depends on the width W interfere with each other, and a target thickness longitudinal vibration cannot be obtained. On the other hand, if the width W of the piezoelectric body is less than a predetermined value with respect to the thickness T, the piezoelectric body may be too thin and complex vibration modes will interfere with each other, and a target thickness longitudinal vibration cannot be obtained. Therefore, a desirable value exists with respect to a ratio W/T between the thickness T and width W of a piezoelectric body. Accordingly, if the ratio W/T of a piezoelectric body is larger than the aforementioned desirable value, in general “sub-dicing is performed” to provide a groove that penetrates through a part or all of the piezoelectric body.

### SUMMARY OF THE INVENTION

### Technical Problem

Although the object of sub-dicing that is performed to prevent the above described interference is to divide an piezoelectric body, if electrodes that correspond to an piezoelectric body are separated, the same voltage cannot be applied to the respective electrodes that correspond to the adjacent piezoelectric bodies, and aperture control for ultrasound beams cannot be performed. Interconnects that electrically connect the electrodes are required in order to enable aperture control of ultrasound beams, and the interconnection structure may become complicated depending on the arrangement of the electrodes and the like. Therefore, as an ultrasound probe that enables aperture control for ultrasound beams, a structure is desirable in which piezoelectric bodies are separated with electrical conduction between electrodes that correspond to the adjoining piezoelectric bodies retained. In general, several hundred piezoelectric bodies are arranged in the array direction in a 1-D (one-dimensional) array ultrasound probe, and in the case of 1.25-D to 1.75-D array ultrasound probes it is necessary to also arrange from several to several tens of piezoelectric bodies in the short-axis direction, and in some cases the total number of piezoelectric bodies reaches several thousand. Consequently, in 1.25-D to 1.75-D array ultrasound probes, the interconnection structure that electrically connects the electrodes becomes more complicated.

An object of the present invention is to provide an ultrasound probe capable of performing highly reliable aperture control for ultrasound beams while limiting interference caused by width vibrations that result from dividing a piezoelectric body.

### Solution to Problem

One aspect of the present invention provides an ultrasound probe including a laminated body that has: a piezoelectric body having a predetermined thickness in a first direction; a first electrode and a second electrode that face each other so as to sandwich the piezoelectric body in the first direction; an intermediate layer that is electrically connected with the second electrode and is provided on an opposite side to the piezoelectric body with respect to the second electrode; and a third electrode that faces the second electrode with the intermediate layer sandwiched therebetween, and extends in a second direction that is orthogonal to the first direction; wherein: a plurality of the first electrodes and the second electrodes are arranged at predetermined intervals in the second direction, respectively; a plurality of the laminated bodies are arranged in a third direction that is orthogonal to the first direction and to the second direction, respectively; and a first groove that penetrates through the first electrode, the piezoelectric body, the second electrode and a part of the intermediate layer and extends in the second direction is formed in the laminated body.

### Advantageous Effects of Invention

According to the ultrasound probe of the present invention, by adopting a sub-diced configuration in which an intermediate layer is provided between a plurality of piezoelectric bodies arranged at predetermined intervals in a second direction and a third electrode that controls the number of piezoelectric bodies that are driven (aperture), and in which a first groove that extends in a second direction penetrates as far as a part of the intermediate layer, electrical connections between the plurality of piezoelectric bodies and the third electrode can be realized with a high degree of reliability that is not affected by the finishing accuracy or component variations when manufacturing the ultrasound probe. According to this configuration, aperture control of the plurality of piezoelectric bodies arranged in the second direction can be performed by means of the third electrode that extends in the second direction.

### BRIEF DESCRIPTION OF THE DRAWINGS

FIG. 1 is a perspective view that illustrates an ultrasound probe according to Embodiment 1 of the present invention;

FIG. 2 is a cross-sectional view obtained by cutting the ultrasound probe of Embodiment 1 that is constituted by a compound piezoelectric structure at third electrode 11-1 along a second direction (short-axis direction);

FIG. 3 illustrates a compound piezoelectric structure in a case where “piezoelectric phase width Wp<<second electrode width We”;

FIG. 4 illustrates a compound piezoelectric structure in a case where “piezoelectric phase width Wp>second electrode width We”;

FIG. 5 is a cross-sectional view of an ultrasound probe obtained by cutting the ultrasound probe at third electrode 11-1, in which intervals between a plurality of intermediate layers arranged in the second direction (short-axis direction) are different in a first direction (thickness direction);

FIG. 6 is a cross-sectional view obtained by cutting the ultrasound probe of Embodiment 1 at third electrode 11-1 along the second direction (short-axis direction);

FIG. 7 is a cross-sectional view obtained by cutting the ultrasound probe of Embodiment 1 at a position of piezoelectric body 3-1 illustrated in FIG. 6 along a third direction (array direction);

FIG. 8 is a cross-sectional view obtained by cutting the ultrasound probe of Embodiment 1 at a position of piezoelectric body 3-1 in the second direction (short-axis direction) along the third direction (array direction);

FIG. 9 is a cross-sectional view obtained by cutting the ultrasound probe of Embodiment 1 at third electrode 11-1 along the second direction (short-axis direction);

FIG. 10 is a perspective view illustrating an intermediate layer having a portion that is formed across a plurality of second electrodes;

FIG. 11 is a schematic drawing of a laminated body when piezoelectric bodies have been laminated on an intermediate layer having a portion that is formed across a plurality of second electrodes;

FIG. 12 illustrates a configuration example of an intermediate layer having a portion that is formed across a plurality of second electrodes;

FIG. 13 illustrates a configuration example of an intermediate layer having a portion that is formed across a plurality of second electrodes;

FIG. 14 is a perspective view that illustrates a configuration example of a double-sided FPC;

FIG. 15 illustrates a configuration example of a transmitting circuit for an arbitrary single channel of the ultrasound probe of Embodiment 1 in which piezoelectric body 3 is divided into five parts in the second direction (short-axis direction);

FIG. 16 is a top view of the double-sided FPC as viewed from the piezoelectric body side in the first direction;

FIG. 17 is a cross-sectional view obtained by cutting ultrasound probe 1 at third electrode 11-1 along the second direction (short-axis direction) in a case where switching was performed to connect a signal line and third electrode 11-1;

FIG. 18 is a cross-sectional view obtained by cutting ultrasound probe 1 at a position of piezoelectric body 3-1 along the third direction (array direction);

FIG. 19 is a cross-sectional view obtained by cutting ultrasound probe 1 at third electrode 11-2 along the second direction (short-axis direction) in a case where switching was performed to connect a signal line and third electrode 11-2;

FIG. 20 is a cross-sectional view obtained by cutting ultrasound probe 1 at a position of piezoelectric body 3-3 along the third direction (array direction);

FIG. 21 is a cross-sectional view obtained by cutting ultrasound probe 1 at third electrode 11-3 along the second direction (short-axis direction) in a case where switching was performed to connect a signal line and third electrode 11-3;

FIG. 22 is a cross-sectional view obtained by cutting ultrasound probe 1 at a position of piezoelectric body 3-2 along the third direction (array direction);

FIG. 23 is a perspective view that illustrates an ultrasound probe according to Embodiment 2 of the present invention;

FIG. 24 is a top view of a single-sided FPC as seen from a piezoelectric body side in the first direction;

FIG. 25 is a cross-sectional view obtained by cutting ultrasound probe 111 at third electrode 11-1 along the second direction (short-axis direction) in a case where switching was performed to connect a signal line and third electrode 11-1;

FIG. 26 is a cross-sectional view obtained by cutting ultrasound probe 111 at a position of piezoelectric body 3-1 along the third direction (array direction);

FIG. 27 is a cross-sectional view obtained by cutting ultrasound probe 111 at third electrode 11-2 along the second direction (short-axis direction) in a case where switching was performed to connect a signal line and third electrode 11-2;

FIG. 28 is a cross-sectional view obtained by cutting ultrasound probe 111 at a position of piezoelectric body 3-3 along the third direction (array direction);

FIG. 29 is a cross-sectional view obtained by cutting ultrasound probe 111 at third electrode 11-3 along the second direction (short-axis direction) in a case where switching was performed to connect a signal line and third electrode 11-3;

FIG. 30 is a cross-sectional view obtained by cutting ultrasound probe 111 at a position of piezoelectric body 3-2 along the third direction (array direction);

FIG. 31 is a perspective view that illustrates an ultrasound probe according to Embodiment 3 of the present invention;

FIG. 32 is a top view of an intermediate layer and a single-sided FPC as seen from a piezoelectric body side in the first direction;

FIG. 33 is a cross-sectional view obtained by cutting ultrasound probe 121 at third electrode 11-1 along the second direction (short-axis direction) in a case where switching was performed to connect a signal line and third electrode 11-1;

FIG. 34 is a cross-sectional view obtained by cutting ultrasound probe 121 at a position of piezoelectric body 3-1 along the third direction (array direction);

FIG. 35 is a cross-sectional view obtained by cutting ultrasound probe 121 at third electrode 11-2 along the second direction (short-axis direction) in a case where switching was performed to connect a signal line and third electrode 11-2;

FIG. 36 is a cross-sectional view obtained by cutting ultrasound probe 121 at a position of piezoelectric body 3-3 along the third direction (array direction);

FIG. 37 is a cross-sectional view obtained by cutting ultrasound probe 121 at third electrode 11-3 along the second direction (short-axis direction) in a case where switching was performed to connect a signal line and third electrode 11-3;

FIG. 38 is a cross-sectional view obtained by cutting ultrasound probe 121 at a position of piezoelectric body 3-2 along the third direction (array direction); and

FIG. 39 is a perspective view that illustrates electrical interconnections of piezoelectric bodies in a three-dimensional ultrasound transducer array described in U.S. Pat. No. 5,617,865.

### DESCRIPTION OF THE EMBODIMENTS

Embodiments of the ultrasound probe according to the present invention will now be described in detail with reference to the accompanying drawings. In the following description, branch numbers of a reference numeral that is assigned to a component are assigned to distinguish individual elements constituting the relevant component.

### Embodiment 1

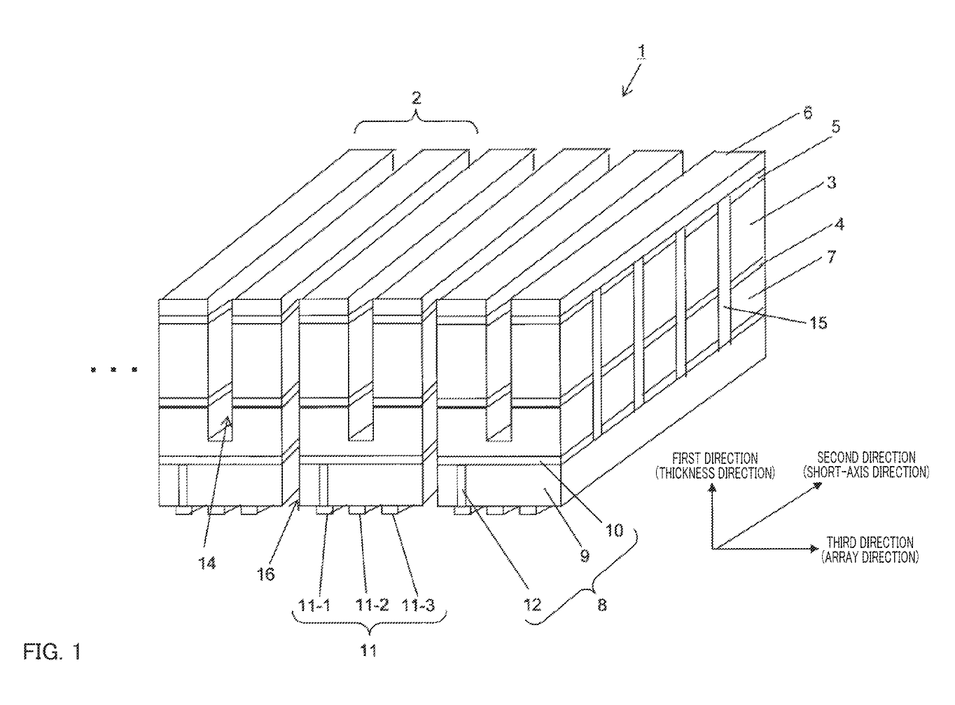

FIG. 1 is a perspective view that illustrates an ultrasound probe according to Embodiment 1 of the present invention. Ultrasound probe 1 shown in FIG. 1 is used in contact with a subject such as a living body. Ultrasound probe 1 is a transducer that directs ultrasound to the subject by applying an electrical signal to piezoelectric body 3 inside ultrasound probe 1, and converts ultrasound reflected from inside subject into an electrical signal using piezoelectric body 3. As shown in FIG. 1, ultrasound probe 1 includes piezoelectric body 3, second electrode 4, first electrode 5, ground layer 6, intermediate layer 7, double-sided FPC 8, third electrode 11, first groove 14, second groove 15, third groove 16, a back surface material (not shown), a plurality of matching layers (not shown), and a lens (not shown).

When a voltage generated by a transmitting circuit (not shown) inside the ultrasound diagnosis apparatus or ultrasound probe 1 is applied between first electrode (ground electrode) 5 and second electrode (signal electrode) 4 that are provided facing each other in a first direction (thickness direction) of piezoelectric body 3 inside ultrasound probe 1, piezoelectric body 3 generates ultrasound. Further, piezoelectric body 3 converts the ultrasound reflected from the subject into an electrical signal. The electrical signal obtained by conversion of the reflected ultrasound by piezoelectric body 3 is sent to a receiving circuit (not shown) inside the ultrasound diagnosis apparatus or ultrasound probe 1 through first electrode 5 and second electrode 4, and processing required for ultrasound diagnosis is performed.

First electrode 5 and second electrode 4 are metallic materials such as gold or silver, and are formed by vapor deposition, plating, sputtering or baking. First electrode 5 and second electrode 4 are formed facing each other so as to sandwich piezoelectric body 3 in the first direction (thickness direction). A plurality of first electrodes 5 and second electrodes 4 are arranged at predetermined intervals in a second direction (short-axis direction).

Piezoelectric body 3 is a material that has a positive piezoelectric effect such that an electrical charge is induced on a surface when stress is applied to the surface (a force is converted to an electrical signal), and an inverse piezoelectric effect such that distortion occurs when an electric field is applied thereto (an electrical signal is converted to a force). The following piezoelectric materials are available as the material of piezoelectric body 3:
piezoelectric ceramics of lead zirconate titanate or lead titanate, or the like;
relaxation-type ferroelectric materials referred to as “relaxors” having a very high relative dielectric constant;
non-lead piezoelectric ceramics or piezoelectric single crystals of barium-base, niobium-base, bismuth-base, or the like;
electrostrictive material of lead magnesium noibate, flexoelectric material, electret material, or the like.
a solid solution single crystal of lead zinc niobate and lead titanate, lead magnesium niobate and lead titanate, or lead indium niobate, lead magnesium niobate, and lead titanate, or the like; and
piezoelectric polymer membranes of polyvinylidene fluoride (PVDF) or the like.

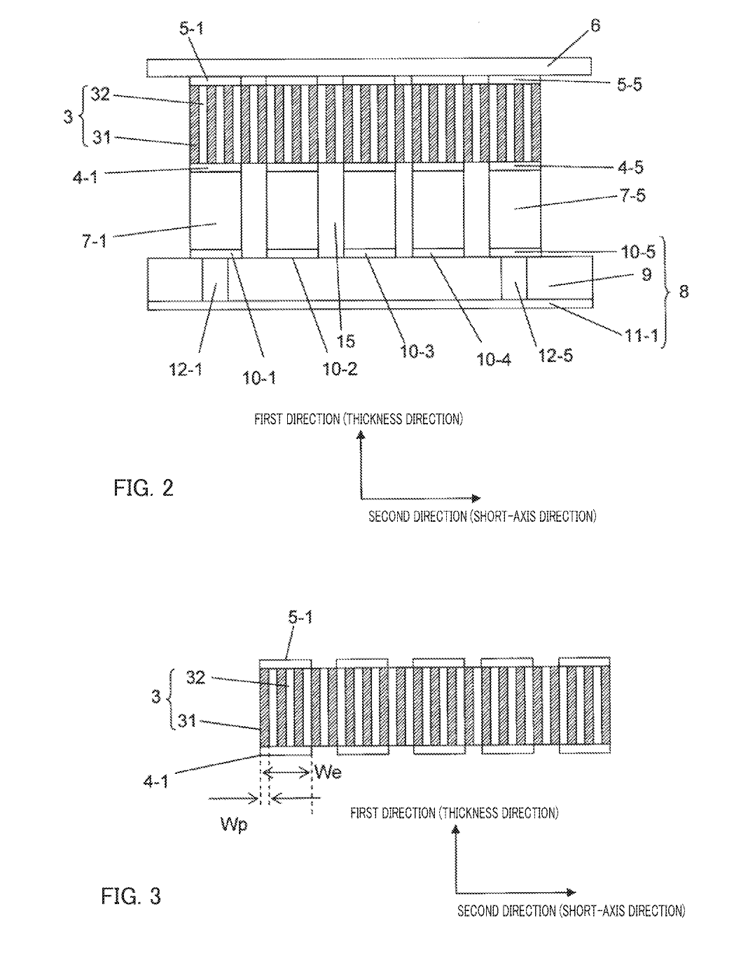

As shown in FIG. 2, piezoelectric body 3 may be configured as a compound piezoelectric structure in which a plurality of piezoelectric phases 31 and resin phases 32 are adjacent to each other in the second direction (short-axis direction). FIG. 2 is a cross-sectional view obtained by cutting ultrasound probe 1 that is constituted by a compound piezoelectric structure at third electrode 11-1 along the second direction (short-axis direction). The same material as that of piezoelectric body 3 is used for piezoelectric phases 31. Further, an insulating material such as phenol resin, epoxy resin, silicone resin or urethane resin is used for resin phases 32. First electrodes 5 and second electrodes 4 face each other at predetermined intervals in the second direction, in a manner such that piezoelectric body 3 is sandwiched therebetween in the first direction. In general, width We of second electrode 4 is from several times to several tens of times larger than a wavelength of the ultrasound that is generated. On the other hand, width Wp of piezoelectric phase 31 is around the same size as the wavelength of the ultrasound. That is, since in general “We>>Wp”, as shown in FIG. 3, no more than one second electrode 4 is connected to a single piezoelectric phase 31. At this time, only piezoelectric body 3 that is sandwiched between first electrode 5 and second electrode 4 vibrates in the thickness longitudinal direction (piezoelectric phases 31 and resin phases 32 vibrate in the thickness longitudinal direction as a united body).

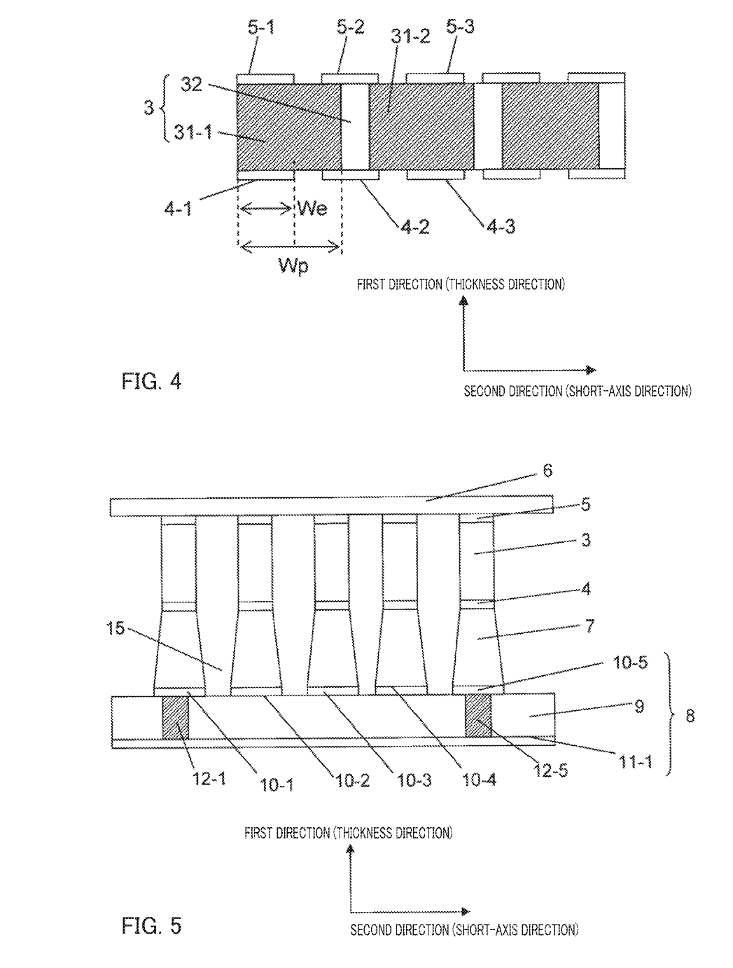

However, as shown in FIG. 4, in a case where width Wp of piezoelectric phase 31 is sufficiently wider than the ultrasound wavelength (Wp>We), one piezoelectric phase 31 is connected to two or more second electrodes 4 and first electrodes 5. In this case, for example, when an electrical signal is applied to only second electrode 4-2 that is shown in FIG. 4, ultrasound is generated from a portion facing second electrode 4-2 of piezoelectric phase 31-1 and piezoelectric phase 31-2. At this time, because piezoelectric phase 31-1 and piezoelectric phase 31-2 vibrate, there is a possibility of “crosstalk” occurring in which an electrical signal arises between second electrode 4-1 and first electrode 5-1 and between second electrode 4-3 and first electrode 5-3 that face piezoelectric phase 31-1 and piezoelectric phase 31-2. It is necessary to reduce such crosstalk because the crosstalk is a noise source. Accordingly, in a case where piezoelectric body 3 is configured as a compound piezoelectric structure in which piezoelectric phases 31 and resin phases 32 are made adjacent in the second direction, as shown in FIG. 2 and FIG. 3, it is necessary to make the width of piezoelectric phase 31 in the second direction narrower than the width of second electrode 4 and first electrode 5.

In addition, piezoelectric body 3 may have a configuration (not shown) in which a plurality of piezoelectric phases 31 and internal electrodes are laminated alternately in the first direction (thickness direction). A metallic material such as nickel or silver-palladium can be used for the internal electrode.

Intermediate layer 7 shown in FIG. 1 is provided on an opposite side to piezoelectric body 3 with respect to second electrode 4, and electrically conducts with second electrode 4 that is a signal electrode. Second groove 15 that extends in a third direction (array direction) penetrates through first electrode 5, piezoelectric body 3, second electrode 4 and intermediate layer 7. A plurality of intermediate layers 7 are arranged at predetermined intervals.

As shown in FIG. 5, a configuration may also be adopted in which intervals between the plurality of intermediate layers 7 that are arranged at predetermined intervals in the second direction (short-axis direction) differ depending on the positions of the respective intermediate layers 7 in the first direction (thickness direction). In the example shown in FIG. 5, although the intervals between intermediate layers 7 on a side close to piezoelectric body 3 are wide, the intervals between intermediate layers 7 narrow as the intervals between intermediate layers 7 approach the side of double-sided FPC 8. A configuration employing a different shape from that illustrated in FIG. 5 may also be adopted.

A composite material in which a conductive filler such as carbon, a silver filler or a copper filler is dispersed in a resin, or a conductive material such as copper, tungsten or tungsten carbide is used as intermediate layer 7.

Examples of materials that may be used as the resin include: thermoplastic resins such as phenol resins, urea resins, melamine resins, epoxy resins, unsaturated polyester resins, silicone resins, and polyurethane resins; general-purpose thermoplastic resins such as polyvinyl chloride resins, polyethylene resins, polypropylene resins, polystyrene resins, ABS resins, acrylonitrile-styrene resins, and acrylic resins; thermoplastic engineering plastics such as nylon 6 resins, nylon 66 resins, polyacetal resins, polycarbonate resins, polyethylene terephthalate resins, modified polyphenylene ether resins, polybutylene terephthalate resins, and ultra-high molecular weight polyethylene resins; and thermoplastic super engineering plastics such as PEEK resins, polyphenylene sulfide resins, polysulfone resins, polyether sulfone resins, polyarylate resins, polyamideimide resins, polyetherimide resins, liquid crystal polymers, polytetrafluoroethylene resins, polychlorotrifluoroethylene resins, and polyvinylidene fluoride resins.

Examples of materials that may be used as the conductive filler include: carbon-based fillers such as carbon black, graphite, carbon fiber, carbon nanotube and graphene; metal-based fillers such as silver particles, copper particles, nickel particles, aluminum fiber, stainless steel fiber, and silver-coated glass beads; metal oxide fillers such as tin oxide (antimony doped), zinc oxide (aluminum doped) and indium oxide (tin doped); and conductive polymer-based fillers such as polyaniline particles and polypyrrole particles.

It is known that in a case where the acoustic impedance of intermediate layer 7 is less than the acoustic impedance of piezoelectric body 3, the thickness resonance frequency of piezoelectric body 3 is a half-wavelength resonance mode. Further, it is known that if the acoustic impedance of intermediate layer 7 is equal to or greater than the acoustic impedance of piezoelectric body 3, the thickness resonance frequency of piezoelectric body 3 is a quarter-wavelength resonance mode. Accordingly, in a case where the frequency of ultrasound that ultrasound probe 1 generates is decided, it is necessary to change the thickness of piezoelectric body 3 in accordance with the acoustic impedance of intermediate layer 7. Note that the acoustic impedance is represented by the product of the density and longitudinal wave velocity of the material.

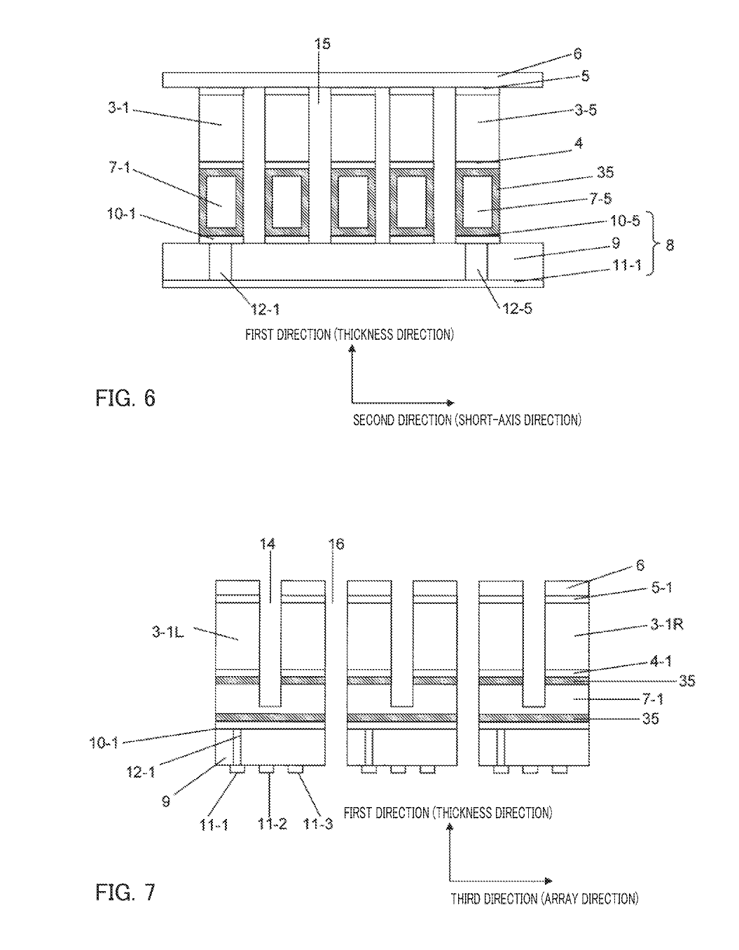

The entire intermediate layer 7 does not necessarily need to have electrical conductivity. That is, as shown in FIG. 6 and FIG. 7, for example, intermediate layer 7 may have a structure in which conductive layer 35 such as gold plating is formed so as to cover the circumference of an insulating resin such as polycarbonate or polypropylene. FIG. 6 is a cross-sectional view obtained by cutting the ultrasound probe of Embodiment 1 at third electrode 11-1 along the second direction (short-axis direction). FIG. 7 is a cross-sectional view obtained by cutting the ultrasound probe of Embodiment 1 at the position of piezoelectric body 3-1 that is shown in FIG. 6 along the third direction (array direction). According to the configuration shown in FIG. 6 and FIG. 7, when a voltage is applied to third electrode 11-1, since the voltage is conducted as far as second electrode 4-1 through conductive section 12-1, fourth electrode 10-1 and conductive layer 35, it is possible to drive both piezoelectric body 3-1L and piezoelectric body 3-1R. The same applies in a case where a voltage is applied to third electrode 11-2 and third electrode 11-3.

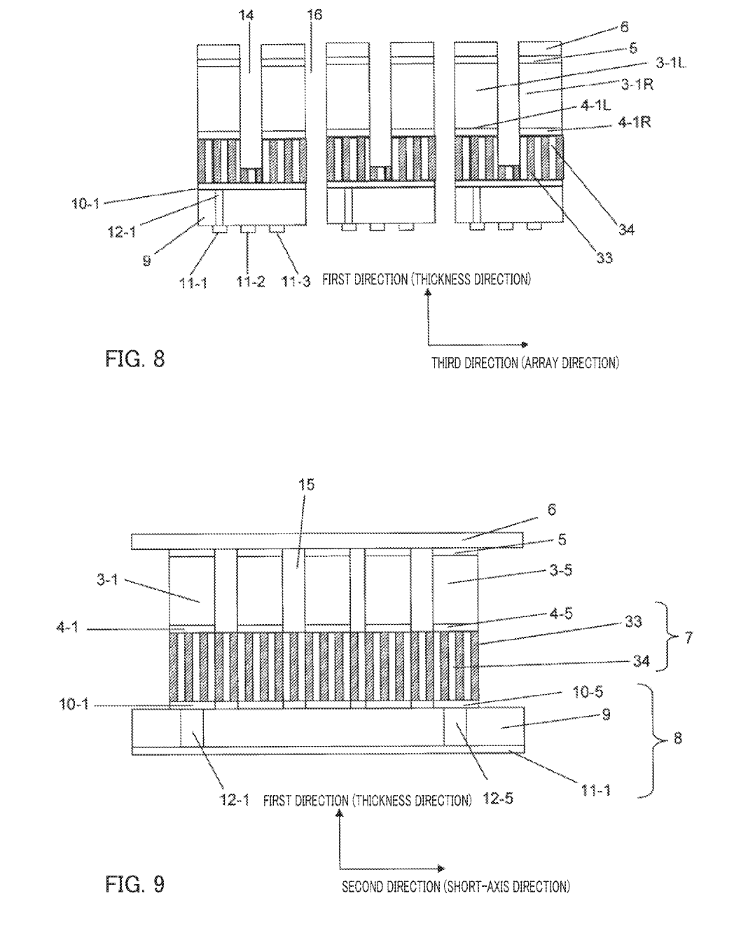

A composite structure in which a plurality of conductor layers 33 and insulator layers 34 are adjacent to each other in the third direction (array direction) may also be adopted as the configuration of intermediate layer 7. FIG. 8 is a cross-sectional view obtained by cutting an ultrasound probe of Embodiment 1 at the position of piezoelectric body 3-1 in the second direction (short-axis direction) along the third direction (array direction). According to the configuration shown in FIG. 8, three conductor layers 33 are arranged in each of the regions that face piezoelectric body 3 and that are adjacent to each other across first groove 14 in the third direction. In a configuration in which at least one conductor layer 33 is arranged in the regions facing piezoelectric body 3, it is possible to drive both piezoelectric body 3-1L and piezoelectric body 3-1R when a voltage is applied to third electrode 11-1, since the voltage is conducted as far as second electrode 4-1L and second electrode 4-1R through conductive section 12-1, fourth electrode 10-1 and conductor layer 33. The same applies in a case where a voltage is applied to third electrode 11-2 and third electrode 11-3.

A laminated structure in which a plurality of conductor layers 33 and insulator layers 34 are arranged alternately side by side in the second direction (short-axis direction) may also be adopted as the configuration of intermediate layer 7. FIG. 9 is a cross-sectional view obtained by cutting an ultrasound probe of Embodiment 1 at third electrode 11-1 along the second direction (short-axis direction). According to the configuration shown in FIG. 9, at least some of insulator layers 34 are arranged in each region that faces a region between second electrodes 4 that are adjacent in the second direction. According to this configuration, when a voltage is applied to third electrode 11-1, the voltage is conducted as far as second electrode 4-1 and second electrode 4-5 through conductive section 12-1 and conductive section 12-5, fourth electrode 10-1 and fourth electrode 10-5, and conductor layers 33 that are electrically connected to fourth electrodes 10-1 and 10-5. On the other hand, fourth electrode 10-1 and fourth electrode 10-5 are not electrically continuous with second electrode 4-2, second electrode 4-3 and second electrode 4-4 due to the presence of insulator layers 34. Consequently, when a voltage is applied to third electrode 11-1, only piezoelectric body 3-1 and piezoelectric body 3-5 are driven. The same applies in a case where a voltage is applied to third electrode 11-2 and third electrode 11-3.

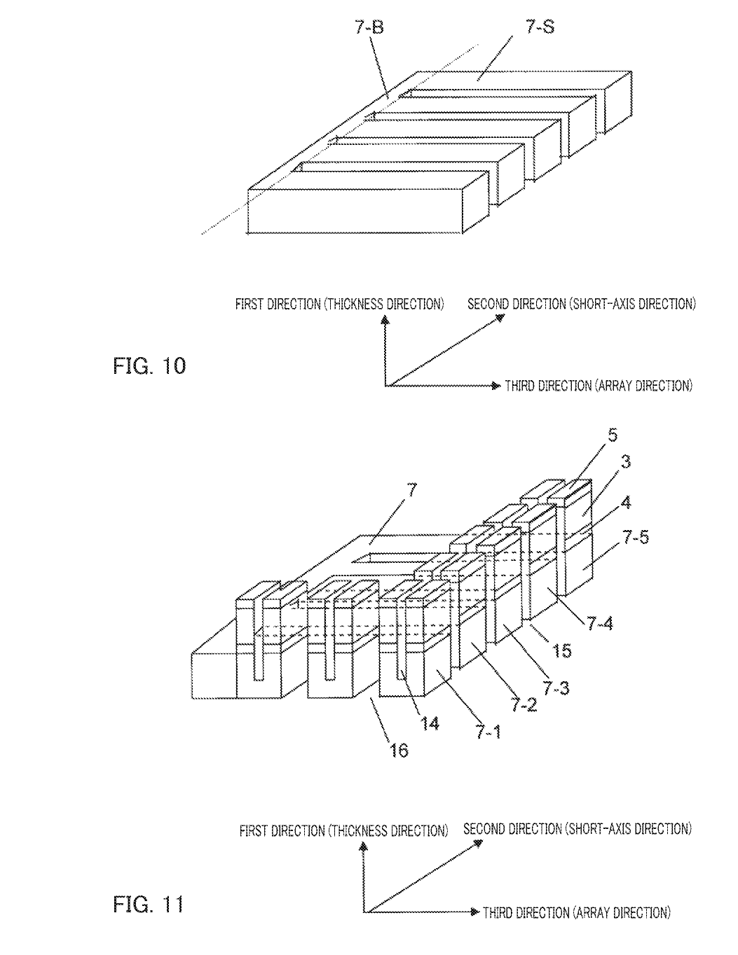

As shown in FIG. 10, intermediate layer 7 may also have a configuration that includes a portion that is formed across a plurality of second electrodes 4. Note that, to simplify the description it is assumed that intermediate layer 7 is formed of the above described conductive material. Intermediate layer 7 shown in FIG. 10 includes portions (divided portions) 7-S that have been divided in the second direction (short-axis direction), and portion (continuous portion) 7-B that is continuous along the second direction. A laminated body in which piezoelectric bodies 3 and the like are laminated on intermediate layer 7 shown in FIG. 10 is shown in FIG. 11. If third grooves 16 were not provided, all second electrodes 4 would be in an electrically connected state through continuous portion 7-B of intermediate layer 7, and hence it would not be possible to select piezoelectric body 3 that is driven when a voltage is applied to third electrode 11 (not shown in FIG. 11). However, by forming third grooves 16, even though continuous portion 7-B of intermediate layer 7 is present, divided portions 7-S and continuous portion 7-B of intermediate layer 7 are electrically separated. Thus, as shown in FIG. 10, even when intermediate layer 7 has continuous portion 7-B that is formed continuously over a plurality of second electrodes 4, it is possible to divide intermediate layer 7 in both the second direction and third direction when manufacturing ultrasound probe 1. Accordingly, it is also possible to select piezoelectric body 3 to be driven, by controlling third electrode 11 that applies a voltage.

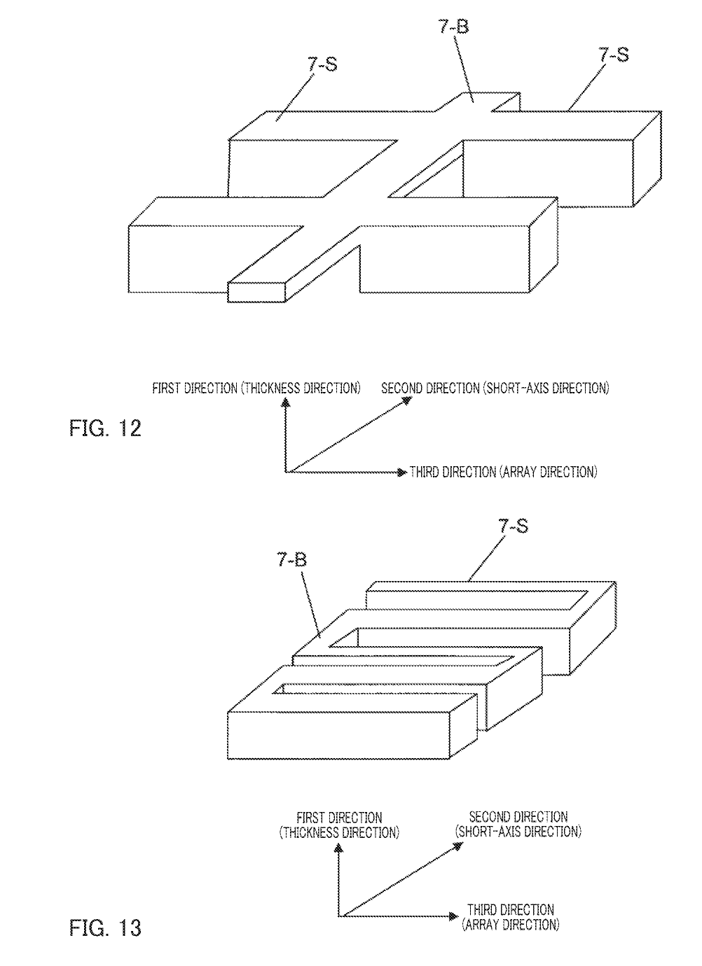

As shown in FIG. 12, intermediate layer 7 may also have a configuration in which there are connections at some parts in the second direction, and in which a connection portion in the first direction (thickness direction) is smaller than intermediate layer 7-S. For example, if the width of intermediate layer 7-B in the third direction (array direction) is narrower than third groove 16 shown in FIG. 11, intermediate layer 7-B will also be removed when forming third groove 16. Consequently, it is possible to divide intermediate layer 7 both in the second direction and third direction. Further, as shown in FIG. 13, all connecting portions of intermediate layer 7 need not necessarily be connected in the second direction, and the same situation as described above applies in the case of a configuration in which only some of the connecting portions are connected. A configuration other than the above described configuration may also be adopted as long as it is possible to divide intermediate layer 7 both in the second direction and third direction when manufacturing ultrasound probe 1.

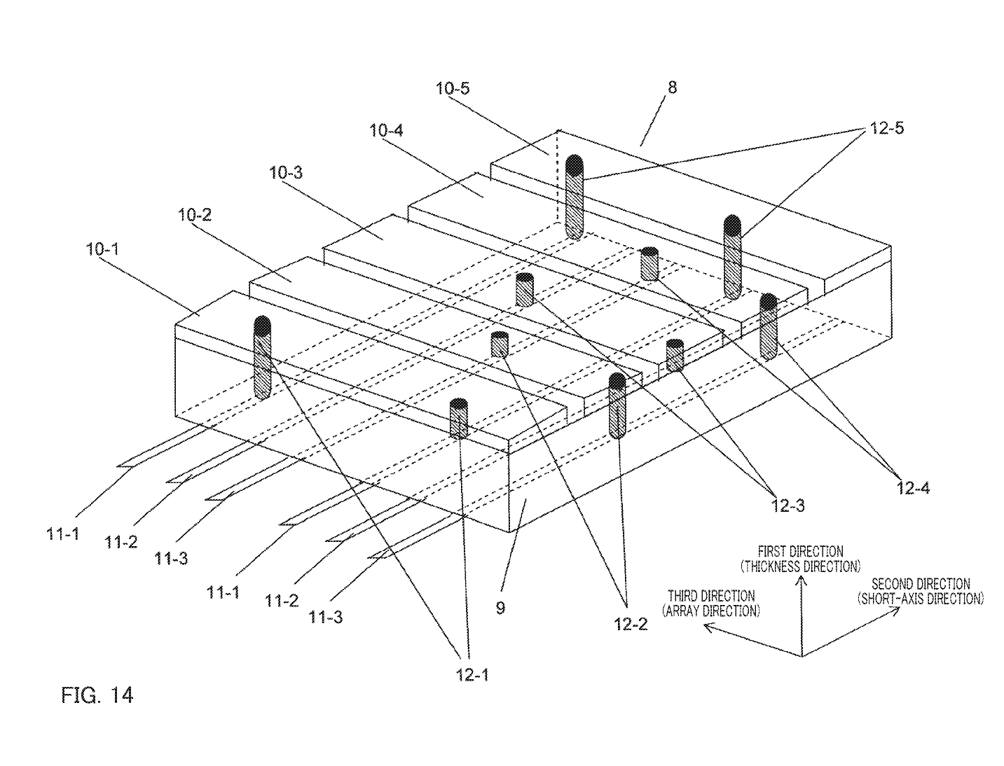

As shown in FIG. 14, double-sided FPC 8 is used in ultrasound probe 1 of the present embodiment. Double-sided FPC 8 includes third electrode 11, fourth electrode 10, conductive section 12 and insulating layer 9. A plurality of fourth electrodes 10 are arranged at predetermined intervals in the second direction (short-axis direction).

In ultrasound probe 1 shown in FIG. 1, a configuration is adopted in which three third electrodes 11 (third electrode 11-1, third electrode 11-2 and third electrode 11-3) per channel extend in the second direction. Third electrodes 11-1 to 11-3 are arranged for each channel pitch in the third direction (array direction). The configuration is such that third electrodes 11-1 to 11-3 are not electrically connected to each other. Third electrode 11 and fourth electrode 10 face each other with insulating layer 9 sandwiched therebetween. Third electrode 11 and fourth electrode 10 are selectively electrically connected through conductive section 12.

According to the example illustrated in FIG. 14, third electrode 11-1 is electrically connected to fourth electrode 10-1 and fourth electrode 10-5 through conductive section 12-1 and conductive section 12-5. However, third electrode 11-1 is not electrically connected to fourth electrodes 10-2 to 10-4. Third electrode 11-2 is electrically connected to fourth electrode 10-3 through conductive section 12-3. However, third electrode 11-2 is not electrically connected to fourth electrodes 10-1, 10-2, 10-4 and 10-5. Third electrode 11-3 is electrically connected to fourth electrode 10-2 and fourth electrode 10-4 through conductive section 12-2 and conductive section 12-4. However, third electrode 11-3 is not electrically connected to fourth electrodes 10-1, 10-3 and 10-5.

Since a component in which third electrode 11, insulating layer 9 and fourth electrode 10 are laminated in advance is commercially available, it is generally the most convenient method to use such a component as double-sided FPC 8. However, a similar structure as double-sided FPC 8 may also be formed using a copper foil film as the material of third electrode 11 and fourth electrode 10, and a polyimide film or a polyester film or the like as insulating layer 9.

Conductive sections 12 are formed by through-holes obtained by boring holes by drilling or the like at required places in double-sided FPC 8 as the component in which third electrode 11, insulating layer 9 and fourth electrode 10 are laminated, and then executing metallic plating or the like around the circumference of the holes, or by a configuration referred to as a “filled via” that is formed by filling portions at which holes are formed with conductive material. Electrical continuity between third electrodes 11 and fourth electrodes 10 can be selectively established by means of conductive sections 12.

In FIG. 1, a configuration is illustrated in which four second grooves 15 that penetrate through first electrode 5, piezoelectric body 3, second electrode 4, and intermediate layer 7 are provided in the second direction (short-axis direction) to thereby divide first electrode 5, piezoelectric body 3, second electrode 4, and intermediate layer 7 into five parts. For example, in a configuration using intermediate layer 7 shown in FIG. 6 or FIG. 7 and FIG. 8, second grooves 15 penetrate through first electrode 5, piezoelectric body 3, second electrode 4, and intermediate layer 7. Apart from the foregoing configuration, for example, in a case of using piezoelectric body 3 shown in FIG. 2, a configuration may be adopted in which second groove 15 penetrates through intermediate layer 7. Further, in a case of using intermediate layer 7 shown in FIG. 9, a configuration may be adopted in which second groove 15 penetrates through only first electrode 5, piezoelectric body 3, and second electrode 4. Second groove 15 is commonly formed by machining using a dicer, and may also be formed using a laser or the like. The formed Second groove 15 is filled with an insulating material such as epoxy resin or silicone resin.

Ground layer 6 electrically connects with first electrode 5 and extends in the second direction (short-axis direction). Ground layer 6 electrically connects with a ground line (not shown) of a transmitting circuit or receiving circuit. A conductive material such as copper foil or a single-sided FPC material in which copper foil and a polyimide film or the like are laminated is favorably used as the material of ground layer 6.

Third groove 16 is a groove for forming laminated body 2 for each channel pitch in the third direction (array direction). Third groove 16 is commonly formed by machining using a dicer, and may also be formed using a laser or the like. The formed third groove 16 is filled with an insulating material such as epoxy resin or silicone resin.

In the first direction (thickness direction), first groove 14 penetrates through first electrode 5, piezoelectric body 3, second electrode 4, and a part of intermediate layer 7 which constitute laminated body 2, and extends in the second direction (short-axis direction). First groove 14 is commonly formed by machining using a dicer, and may also be formed using a laser or the like. The formed first groove 14 is filled with an insulating material such as epoxy resin or silicone resin.

When the width of piezoelectric body 3 in the third direction is for example taken to be 0.18 mm and the thickness of piezoelectric body 3 in the first direction is for example taken to be 0.15 mm, W/T=0.18/0.15=1.2 for piezoelectric body 3. If only one first groove 14 is formed in the center of piezoelectric body 3 in the third direction using a dicer having a blade width of 0.02 mm, the width of one piezoelectric body 3 in the third direction will be (0.18−0.02)/2=0.08 mm. Since the thickness of piezoelectric body 3 in the first direction will be unchanged and remain 0.15 mm, the ratio W/T for piezoelectric body 3 will be 0.53. Thus, the common ratio W/T that is from 0.4 to 0.6 can be satisfied.

Further, although first groove 14 is set and machined so as to penetrate as far as a part of intermediate layer 7, if the thickness of intermediate layer 7 is small, there is a possibility that fourth electrode 10 that is electrically connected to intermediate layer 7 will be cut. While the method of manufacturing ultrasound probe 1 will be described later, it is desirable that the thickness of intermediate layer 7 is 0.01 mm or more.

A back surface material (not shown) may be provided on the opposite side to third electrode 11 with respect to insulating layer 9. The back surface material is used as base material for maintaining the shape of laminated body 2 or when forming laminated body 2 in a convex shape. Further, ultrasound generated with piezoelectric body 3 propagates not only to the living body side but also to the back surface material. Ultrasound that propagates to the back surface material side and is reflected at the boundary between the back surface material and the outside is received by piezoelectric body 3, and such ultrasound cannot be distinguished from ultrasound reflected by the living body. Therefore, generally, the material that is used as the back surface material has a function that attenuates ultrasound that propagates to the back surface material side as much as possible so that, even if ultrasound is reflected, the reflected ultrasound does not affect a reflected signal from the living body. Ferrite rubber, urethane resin, epoxy resin or the like is used as the material of the back surface material. In addition, a composite material obtained by mixing the aforementioned materials with a metal powder filler such as iron or tungsten powder, a metal-oxide filler such as alumina, or microballoons or the like may also be used. Further, the same material as intermediate layer 7 may also be used.

A matching layer (not shown) may be provided on an opposite side to piezoelectric body 3 with respect to first electrode 5. A matching layer is used to match the acoustic impedance of piezoelectric body 3 and the living body. It is common to laminate a plurality of matching layers so that the acoustic impedance gradually becomes lower from piezoelectric body 3 towards the living body. Although in the example shown in FIG. 1 a configuration is adopted in which a plurality of first electrodes 5 are arranged at predetermined intervals and ground layer 6 that extends in the second direction (short-axis direction) is provided on the top face thereof, a configuration may also be adopted in which a matching layer having electrical conductivity is electrically connected to first electrode 5, and ground layer 6 is provided on an opposite side to first electrode 5 with respect to the matching layer. A configuration may also be adopted in which a plurality of matching layers are laminated on ground layer 6. A composite material obtained by mixing a filler such as a metal or metal-oxide filler with a ceramic-based material such as a free-cutting ceramic material, silicon, a graphite-based material, epoxy resin or phenol resin or the like, a plastic such as polycarbonate, polystyrene or polyimide, or a rubber-based material such as urethane rubber, nitrile rubber (NBR) or chloroprene rubber or the like is used as the material of the matching layer.

A lens (not shown) is used to focus ultrasound generated by piezoelectric body 3 inside the living body. The lens is formed in a convex shape or a concave surface shape in accordance with the longitudinal wave velocity of the material thereof. In general, taking into account the adhesive properties with respect to a living body, a convex lens is formed on the matching layer side that is the opposite side to piezoelectric body 3 using silicone resin or the like whose longitudinal wave velocity is higher than that of water (living body). Note that, a configuration may also be adopted that does not use a lens and in which ultrasound is focused inside the living body by forming ultrasound probe 1 in a concave surface shape with regard to the second direction (short-axis direction).

(Method for Manufacturing Ultrasound Probe 1 of Embodiment 1)

Hereunder, an example of a method for manufacturing ultrasound probe 1 shown in FIG. 1 is described. As shown in FIG. 14, double-sided FPC 8 includes third electrodes 11, fourth electrodes 10, insulating layer 9 and conductive sections 12. Fourth electrode 10 is previously divided into five parts at predetermined intervals in the second direction. Third electrodes 11 are configured so that three third electrodes 11 extend in the second direction (short-axis direction) with respect to a single laminated body 2 shown in FIG. 1. As shown in FIG. 10, intermediate layer 7 is previously worked into a shape in which a part of a rectangular parallelepiped intermediate layer is divided into five parts in the second direction (short-axis direction). First electrode 5 and second electrode 4 are previously formed facing piezoelectric body 3 in the first direction (thickness direction) from both sides.

(1) First, the back surface material is fixed on a fixing base using wax or the like.

(2) Next, double-sided FPC 8, intermediate layer 7, and piezoelectric body 3 are laminated in that order on the back surface material. When laminating intermediate layer 7 on double-sided FPC 8, alignment is performed so that fourth electrode 10 and intermediate layer 7 face each other. The respective materials are bonded and cured using an adhesive such as epoxy resin.

(3) After the materials are bonded and cured, a dicer is used to form four second grooves 15 that penetrate through first electrode 5, piezoelectric body 3, and second electrode 4 along the third direction (array direction) so as to match the positions at which intermediate layer 7 was divided. Since intermediate layer 7 is machined in advance so as to be divided into five parts, the structure is one in which second grooves 15 penetrate through first electrode 5, piezoelectric body 3, second electrode 4, and intermediate layer 7.

(4) Next, second grooves 15 are filled with epoxy resin, and ground layer 6 and a plurality of matching layers are sequentially laminated on first electrode 5 and bonded and cured.

(5) Next, using a dicer, third grooves 16 that penetrate from the matching layers to a part of the back surface material are provided along the second direction (short-axis direction) to form a plurality of laminated bodies 2, and first grooves 14 that penetrate from the matching layers to a part of intermediate layer 7 are formed in each laminated body 2. Further, although some parts of intermediate layer 7 have connecting portions in the second direction, when forming third groove 16, the relevant parts are electrically separated from adjoining laminated body 2.

(6) Next, after detaching the back surface material from the fixing base, the structure made from a plurality of laminated bodies 2 formed on the back surface material is formed into a convex or linear shape, and thereafter first grooves 14 and third grooves 16 are filled with silicone resin or the like. Thereafter, a lens is attached onto the top face of the matching layers using a silicon-based adhesive or the like.

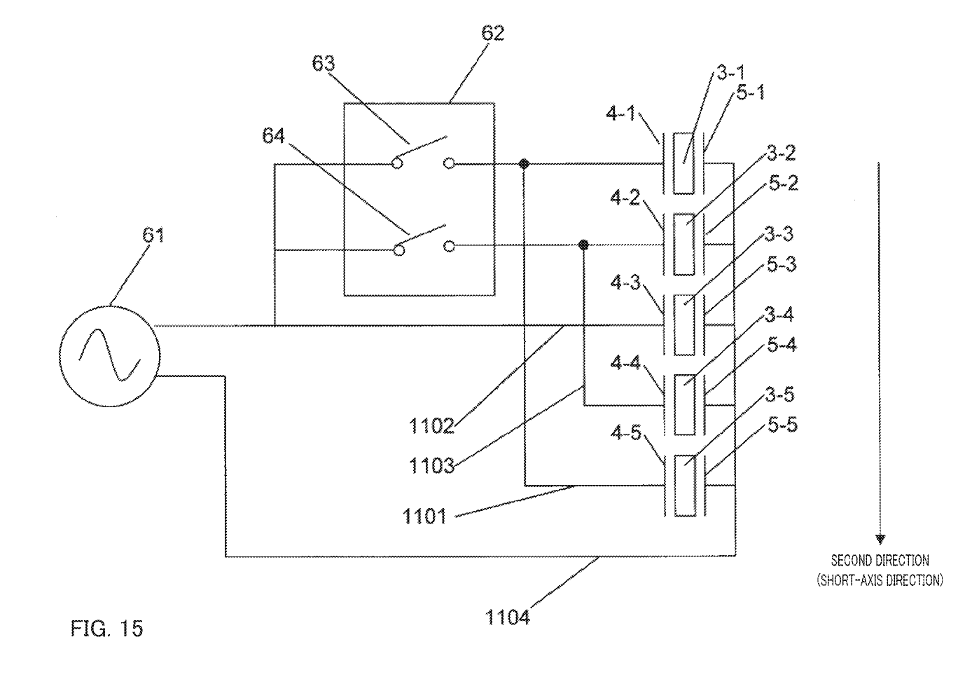

(7) Each of third electrodes 11-1 to 11-3 and ground layer 6 are electrically connected to signal lines 1101 to 1103 and ground line 1104 as shown in FIG. 15 to complete the construction of ultrasound probe 1.

FIG. 15 illustrates a configuration example of a transmitting circuit for an arbitrary single channel of ultrasound probe 1 of Embodiment 1 is which piezoelectric body 3 is divided into five parts in the second direction (short-axis direction). Signal lines 1101 to 1103 that originate from transmitting circuit 61 are connected to second electrodes 4 through switching circuit 62 constituted by a multiplexer or the like, or are directly connected to second electrodes 4. Switching circuit 62 includes switch 63 and switch 64. In the example shown in FIG. 15, second electrode 4-3 is connected to signal line 1102. Second electrode 4-1 and second electrode 4-5 are connected to switch 63 through signal line 1101. Second electrode 4-2 and second electrode 4-4 are connected to switch 64 through signal line 1103. Further, ground line 1104 that originates from transmitting circuit 61 is connected to first electrode 5.

In order to focus at a near position from ultrasound probe 1, switch 63 and switch 64 are turned off since a large aperture is not required. At such time, transmitting circuit 61 is connected to only second electrode 4-3 through signal line 1102, and ultrasound is generated only from piezoelectric body 3-3. In order to focus at a deeper position from ultrasound probe 1, only switch 64 is turned on. At such time, transmitting circuit 61 is connected to second electrode 4-3 through signal line 1102, and because switch 64 has been turned on, transmitting circuit 61 is connected to both second electrode 4-2 and second electrode 4-4 through signal line 1103. As a result, ultrasound is generated from piezoelectric body 3-2, piezoelectric body 3-3 and piezoelectric body 3-4. At such time, the short-axis aperture expands in comparison to when generating ultrasound from only piezoelectric body 3-3, and an ultrasound beam can be focused at a deeper position. In order to focus at an even deeper position from ultrasound probe 1, both switch 63 and switch 64 are turned on. At such time, because switch 63 has been turned on, transmitting circuit 61 is connected to both second electrode 4-1 and second electrode 4-5 through signal line 1101. That is, ultrasound is generated from all five piezoelectric bodies 3-1 to 3-5. In this case, the short-axis aperture expands more than when only piezoelectric body 3-3 or piezoelectric bodies 3-2 to 3-4 generate ultrasound, and an ultrasound beam can be focused at a deeper position.

By controlling the short-axis aperture (number and positions of the piezoelectric bodies that are driven) of the piezoelectric bodies in the second direction (short-axis direction) in this manner, an ultrasound beam can be focused with respect to a plurality of depths inside a living body. Further, a lateral resolution and a longitudinal resolution of an ultrasound diagnostic image are improved in comparison to a 1-D array ultrasound probe. Note that, in particular, in the case of 1.25-D to 1.75-D array ultrasound probes, since there is an extremely large number of piezoelectric bodies 3, switching circuit 62 constituted by a multiplexer or the like is contained inside ultrasound probe 1 as shown in FIG. 15.

(Operations of Ultrasound Probe 1 of Embodiment 1)

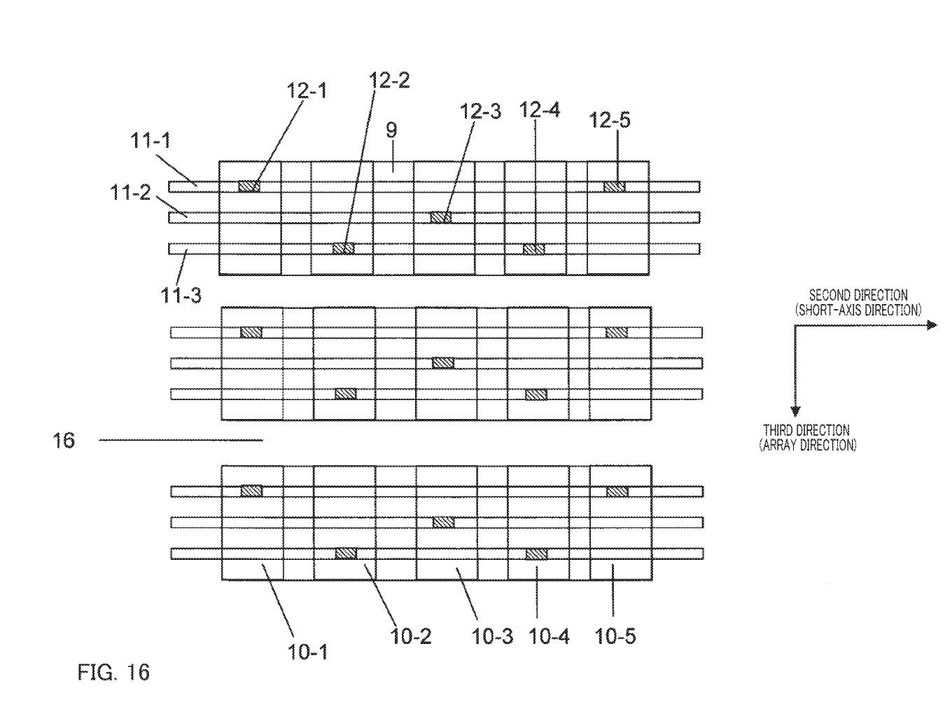

Operations of ultrasound probe 1 of Embodiment 1 will now be described using FIG. 16 to FIG. 22.

A voltage generated by a transmitting circuit (not shown) inside the ultrasound diagnosis apparatus or ultrasound probe 1 is applied through a signal line (not shown) and a ground line (not shown) to piezoelectric body 3. Third electrode 11 to be connected to the signal line when applying the voltage to piezoelectric body 3 is selected by a switching circuit (not shown) such as a multiplexer.

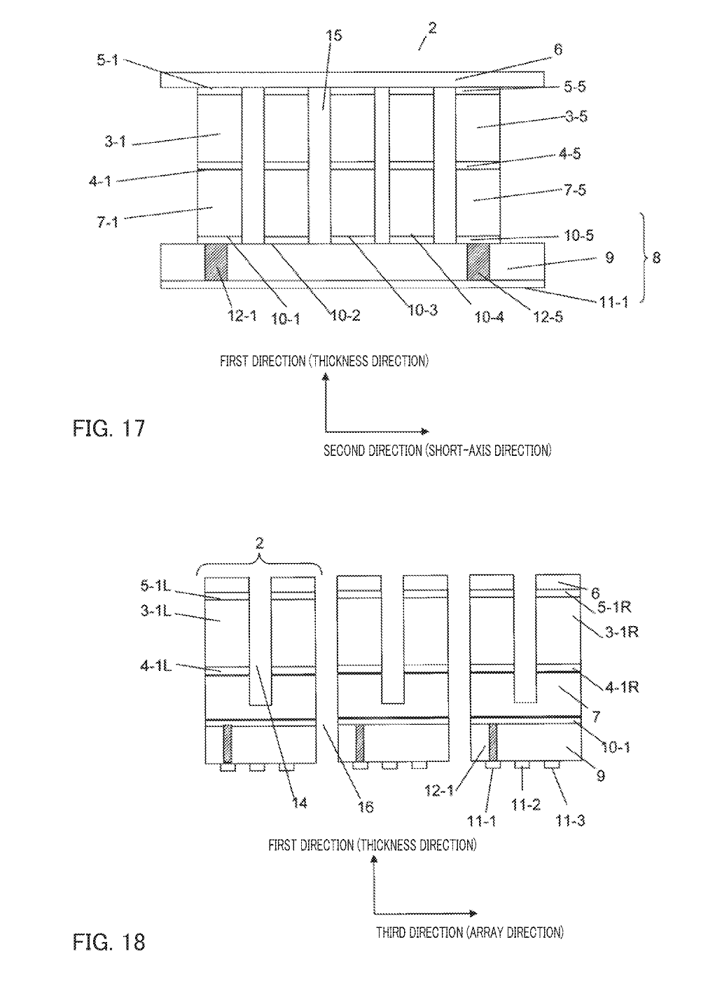

FIG. 17 is a cross-sectional view obtained by cutting ultrasound probe 1 at third electrode 11-1 along the second direction (short-axis direction) in a case where switching was performed to connect a signal line and third electrode 11-1. In the state illustrated in FIG. 17, third electrode 11-1 is electrically connected to fourth electrode 10-1 and fourth electrode 10-5 through conductive section 12-1 and conductive section 12-5. Fourth electrode 10-1 and fourth electrode 10-5 are electrically connected to second electrode 4-1 and second electrode 4-5 through intermediate layer 7-1 and intermediate layer 7-5 to which fourth electrode 10-1 and fourth electrode 10-5 are electrically connected, respectively. On the other hand, the ground line is electrically connected to ground layer 6 that is electrically connected to first electrode 5. In this state, since the voltage applied through the signal line is only applied to second electrode 4-1 and second electrode 4-5, ultrasound is generated only at piezoelectric body 3-1 and piezoelectric body 3-5, and ultrasound is not generated at the other piezoelectric bodies 3-2 to 3-4.

FIG. 18 is a cross-sectional view obtained by cutting ultrasound probe 1 at the position of piezoelectric body 3-1 along the third direction (array direction). One first groove 14 that penetrates through first electrode 5, piezoelectric body 3 and second electrode 4 and a part of intermediate layer 7 is provided in laminated body 2, and consequently piezoelectric body 3 is divided in two in the third direction (array direction). In the configuration shown in FIG. 18, first electrodes 5, piezoelectric bodies 3, and second electrodes 4 on the left and right sides obtained as a result of dividing piezoelectric body 3 in two are referred to as first electrodes 5-1L and 5-1R, piezoelectric bodies 3-1L and 3-1R, and second electrodes 4-1L and 4-1R, respectively. An electrical signal that passed through third electrode 11-1 is electrically connected with fourth electrode 10-1 and intermediate layer 7 through conductive section 12-1, and furthermore flows to piezoelectric body 3-1L and piezoelectric body 3-1R through second electrode 4-1L and second electrode 4-1R. In the present embodiment, even though second electrode 4 is divided by first groove 14, because intermediate layer 7 that is not completely divided is interposed between second electrode 4 and third electrode 11, a voltage can be applied to both piezoelectric body 3-1L and piezoelectric body 3-1R. The same also applies in the case of a cross section (not shown) obtained by cutting ultrasound probe 1 at the position of piezoelectric body 3-5, and is not limited to piezoelectric body 3-1. Therefore, when a voltage is applied between third electrode 11-1 and ground layer 6, ultrasound is generated at piezoelectric body 3-1L, piezoelectric body 3-1R, piezoelectric body 3-5L and piezoelectric body 3-5R.

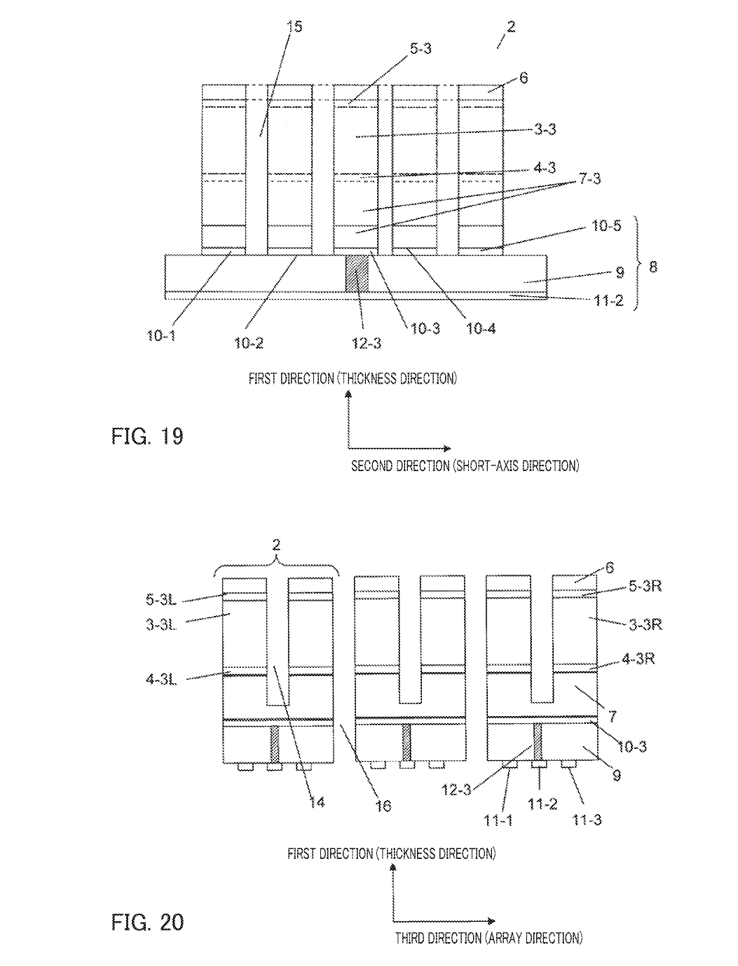

FIG. 19 is a cross-sectional view obtained by cutting ultrasound probe 1 at third electrode 11-2 along the second direction (short-axis direction) in a case where switching was performed to connect a signal line and third electrode 11-2. In the state illustrated in FIG. 19, third electrode 11-2 is electrically connected to fourth electrode 10-3 through conductive section 12-3. Fourth electrode 10-3 is electrically connected to second electrode 4-3 through intermediate layer 7-3 that is electrically connected thereto. On the other hand, the ground line is electrically connected to ground layer 6 that is electrically connected to first electrode 5. In this state, since a voltage applied through the signal line is only applied to second electrode 4-3, ultrasound is generated only at piezoelectric body 3-3, and ultrasound is not generated at the other piezoelectric bodies 3-1, 3-2, 3-4 and 3-5.

FIG. 20 is a cross-sectional view obtained by cutting ultrasound probe 1 at the position of piezoelectric body 3-3 along the third direction (array direction). One first groove 14 that penetrates through first electrode 5, piezoelectric body 3 and second electrode 4 and a part of intermediate layer 7 is provided in laminated body 2, and consequently piezoelectric body 3 is divided in two in the third direction (array direction). In the configuration shown in FIG. 20, first electrodes 5, piezoelectric bodies 3, and second electrodes 4 on the left and right sides obtained as a result of dividing piezoelectric body 3 in two are referred to as first electrodes 5-3L and 5-3R, piezoelectric bodies 3-3L and 3-3R, and second electrodes 4-3L and 4-3R, respectively. An electrical signal that passed through third electrode 11-2 is electrically connected with fourth electrode 10-3 and intermediate layer 7 through conductive section 12-3, and furthermore flows to piezoelectric body 3-3L and piezoelectric body 3-3R through second electrode 4-3L and second electrode 4-3R. In the present embodiment, even though second electrode 4 is divided by first groove 14, because intermediate layer 7 that is not completely divided is interposed between second electrode 4 and third electrode 11, a voltage can be applied to both piezoelectric body 3-3L and piezoelectric body 3-3R. Therefore, when a voltage is applied between third electrode 11-2 and ground layer 6, ultrasound is generated at piezoelectric body 3-3L, piezoelectric body 3-3R.

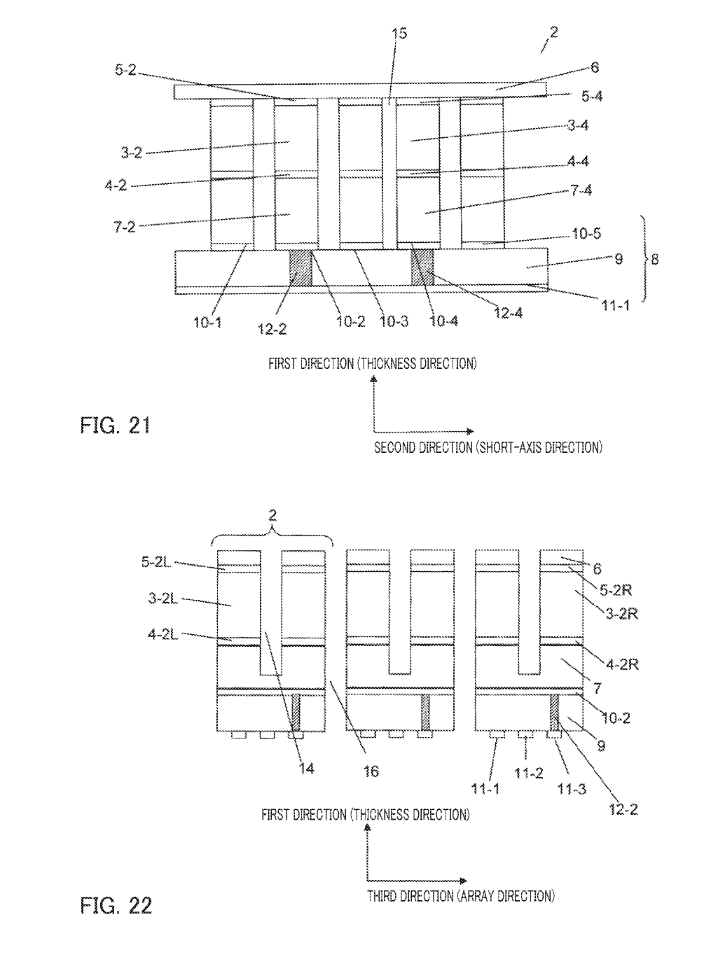

FIG. 21 is a cross-sectional view obtained by cutting ultrasound probe 1 at third electrode 11-3 along the second direction (short-axis direction) in a case where switching was performed to connect a signal line and third electrode 11-3. In the state illustrated in FIG. 21, third electrode 11-3 is electrically connected to fourth electrode 10-2 and fourth electrode 10-4 through conductive section 12-2 and conductive section 12-4. Fourth electrode 10-2 and fourth electrode 10-4 are electrically connected to second electrode 4-2 and second electrode 4-4 through intermediate layer 7-2 and intermediate layer 7-4 to which fourth electrode 10-2 and fourth electrode 10-4 are electrically connected, respectively. On the other hand, the ground line is electrically connected to ground layer 6 that is electrically connected to first electrode 5. In this state, since the voltage applied through the signal line is only applied to second electrode 4-2 and second electrode 4-4, ultrasound is generated only at piezoelectric body 3-2 and piezoelectric body 3-4, and ultrasound is not generated at the other piezoelectric bodies 3-1, 3-3 and 3-5.

FIG. 22 is a cross-sectional view obtained by cutting ultrasound probe 1 at the position of piezoelectric body 3-2 along the third direction (array direction). One first groove 14 that penetrates through first electrode 5, piezoelectric body 3 and second electrode 4 and a part of intermediate layer 7 is provided in laminated body 2, and consequently piezoelectric body 3 is divided in two in the third direction (array direction). In the configuration shown in FIG. 22, first electrodes 5, piezoelectric bodies 3, and second electrodes 4 on the left and right sides obtained as a result of dividing piezoelectric body 3 in two are referred to as first electrodes 5-2L and 5-2R, piezoelectric bodies 3-2L and 3-2R, and second electrodes 4-2L and 4-2R, respectively. An electrical signal that passed through third electrode 11-3 is electrically connected with fourth electrode 10-2 and intermediate layer 7 through conductive section 12-2, and furthermore flows to piezoelectric body 3-2L and piezoelectric body 3-2R through second electrode 4-2L and second electrode 4-2R. In the present embodiment, even though second electrode 4 is divided by first groove 14, because intermediate layer 7 that is not completely divided is interposed between second electrode 4 and third electrode 11, a voltage can be applied to both piezoelectric body 3-2L and piezoelectric body 3-2R. The same also applies in the case of a cross section (not shown) obtained by cutting ultrasound probe 1 at the position of piezoelectric body 3-4, and is not limited to piezoelectric body 3-2. Therefore, when a voltage is applied between third electrode 11-3 and ground layer 6, ultrasound is generated at piezoelectric body 3-2L, piezoelectric body 3-2R, piezoelectric body 3-4L and piezoelectric body 3-4R.

Here, the ultrasound intensity distribution at a time that, with respect to one arbitrary channel, among five piezoelectric bodies 3-1 to 3-5 that include piezoelectric body 3-1, piezoelectric body 3-2, piezoelectric body 3-3, piezoelectric body 3-4 and piezoelectric body 3-5 that are arranged from one end in that order in the second direction (short-axis direction) as shown in FIG. 17, the short-axis aperture of the piezoelectric bodies that are driven is changed in accordance with the following conditions 1 to 3 will now be described:

Condition 1: Drive only piezoelectric body 3-3
Condition 2: Drive piezoelectric bodies 3-2 to 3-4
Condition 3: Drive all piezoelectric bodies (piezoelectric bodies 3-1 to 3-5)

In a case where only the center piezoelectric body 3-3 among the five piezoelectric bodies is driven as indicated by condition 1, the ultrasound intensity shows a high value only in the vicinity of the center of piezoelectric bodies 3 in the second direction (short-axis direction), and the ultrasound intensity gradually decreases as the distance from the center increases. In a case where piezoelectric bodies 3-2 to 3-4 are driven simultaneously as indicated by condition 2, a region in which the ultrasound intensity shows a high value is the vicinity of the center of piezoelectric bodies 3 in the second direction (short-axis direction), and that region is wider in the second direction (short-axis direction) that in the case where only piezoelectric body 3-3 is driven. Similarly, in a case where all piezoelectric bodies 3-1 to 3-5 are driven simultaneously as indicated by condition 3, a region in which the ultrasound intensity shows a high value is the widest in comparison with conditions 1 and 2. Thus, the aperture of an ultrasound beam can be controlled by controlling the short-axis aperture (number and positions of the piezoelectric bodies that are driven).

In a case where first groove 14 penetrates as far as fourth electrode 10, irrespective of similar short-axis aperture control being performed, the ultrasound intensity distribution obtained in the second direction (short-axis direction) for the channel is different to a case where first groove 14 does not penetrate as far as fourth electrode 10.

A significant difference in the ultrasound intensity distribution is not observed when only piezoelectric body 3-3 is driven. However, when piezoelectric body 3-2 to piezoelectric body 3-4 are driven simultaneously, the region in which the ultrasound intensity shows a high value is narrower in the case where first groove 14 penetrates as far as fourth electrode 10. In addition, when all of piezoelectric bodies 3-1 to 3-5 are driven simultaneously, a region in which the ultrasound intensity shows a high value is narrower in the case where first groove 14 penetrates as far as fourth electrode 10, and the ultrasound intensity decreases significantly in regions corresponding to the positions of piezoelectric body 3-1 and piezoelectric body 3-5.

Thus, although forming first groove 14 as far as fourth electrode 10 is considered not to influence the central piezoelectric body 3-3, the other piezoelectric bodies 3-1 to 3-2 and 3-4 to 3-5 can no longer be driven as intended. That is, in a configuration in which first groove 14 penetrates as far as fourth electrode 10, even if short-axis aperture control is performed, an intended ultrasound beam is not obtained particularly at the piezoelectric bodies other than the piezoelectric bodies at the center. However, when a configuration is adopted as in Embodiment 1 in which first groove 14 does not penetrate as far as fourth electrode 10, it is possible to direct a desired ultrasound beam in accordance with the short-axis aperture control.

As described above, according to the present embodiment, since the configuration includes intermediate layer 7 so that first groove 14 for dividing piezoelectric body 3 does not penetrate as far as fourth electrode 10, it is possible to direct a desired ultrasound beam in accordance with the short-axis aperture control. It is thus possible to provide ultrasound probe 1 that is capable of performing highly reliable aperture control with respect to an ultrasound beam while suppressing interference caused by width vibrations that result from dividing piezoelectric body 3.

### Embodiment 2

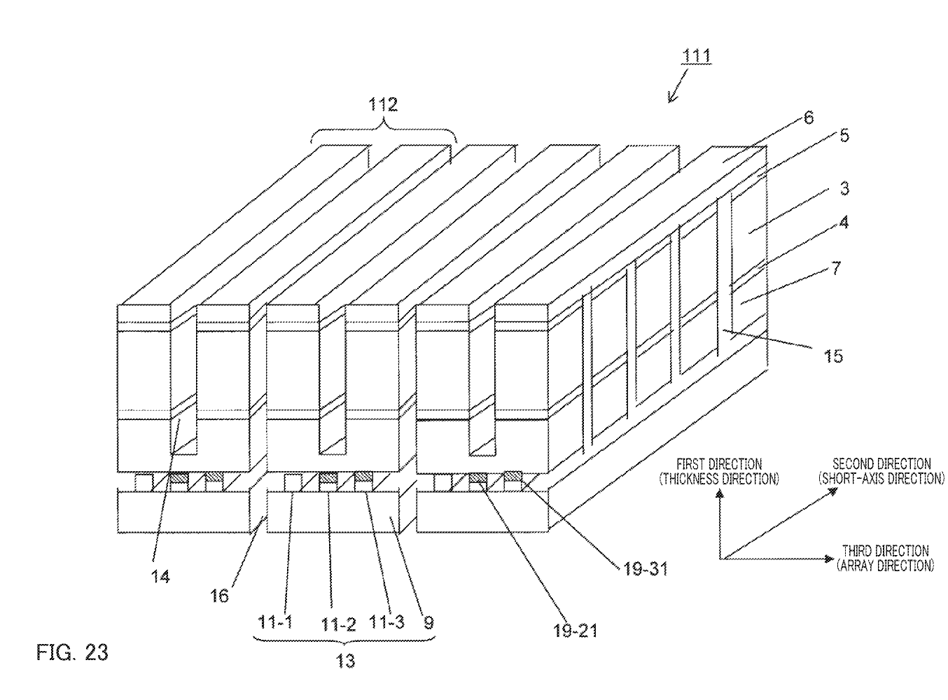

FIG. 23 is a perspective view illustrating an ultrasound probe according to Embodiment 2 of the present invention. Ultrasound probe 111 of Embodiment 2 includes single-sided FPC 13 instead of double-sided FPC 8 that is included in ultrasound probe 1 of Embodiment 1. Other than this difference, ultrasound probe 111 is the same as ultrasound probe 1 of Embodiment 1, and components in FIG. 23 that are common with FIG. 1 are denoted by the same reference numerals as in FIG. 1.

Single-sided FPC 13 includes third electrode 11 that extends in the second direction (short-axis direction) and insulating layer 9. Insulating layer 9 is formed of a polyimide film, polyester film or the like. Similarly to double-sided FPC 8, single-sided FPC 13 is a commercially available component on which third electrode 11 and insulating layer 9 are laminated in advance, and use of the commercially available component is the most convenient way to obtain single-sided FPC 13.

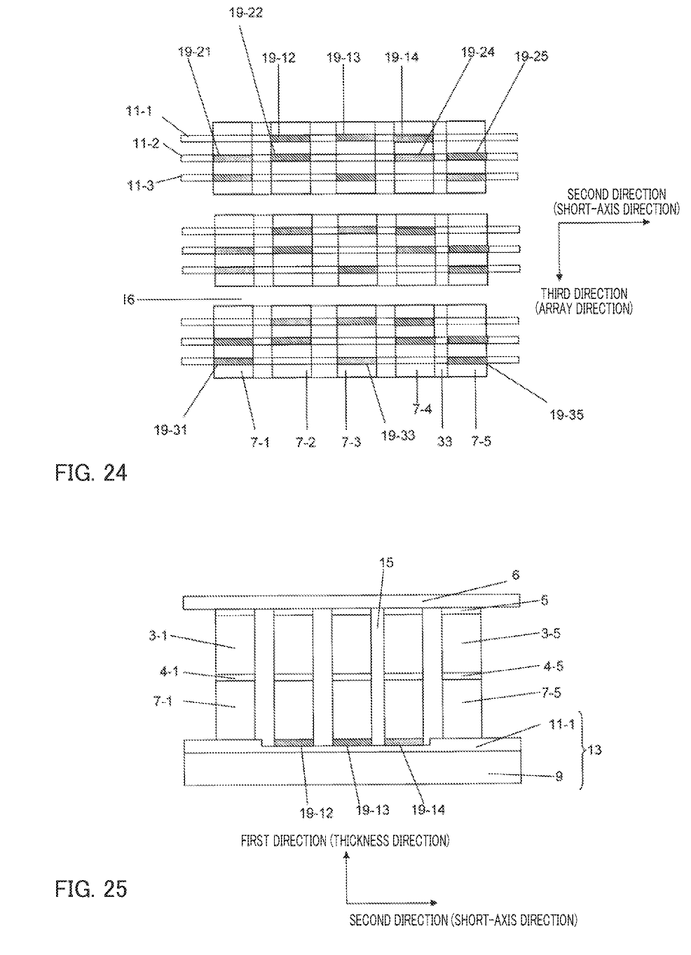

Insulating layers 19 shown in FIG. 23 are provided in the second direction (short-axis direction) on some parts of third electrodes 11 of single-sided FPC 13. An insulating material such as a resist material or a polyimide film or the like is used for insulating layers 19. FIG. 24 shows an example of the arrangement of insulating layers 19. Insulating layer 19-12, insulating layer 19-13 and insulating layer 19-14 shown in FIG. 24 are provided on third electrode 11-1 at positions facing intermediate layer 7-2, intermediate layer 7-3 and intermediate layer 7-4, respectively. Insulating layer 19-21, insulating layer 19-22, insulating layer 19-24 and insulating layer 19-25 are provided on third electrode 11-2 at positions facing intermediate layer 7-1, intermediate layer 7-2, intermediate layer 7-4 and intermediate layer 7-5, respectively. Further, insulating layer 19-31, insulating layer 19-33 and insulating layer 19-35 are provided on third electrode 11-3 at positions facing intermediate layer 7-1, intermediate layer 7-3 and intermediate layer 7-5, respectively.

(Operations of Ultrasound Probe 111 of Embodiment 2)

Operations of ultrasound probe 111 of Embodiment 2 will now be described using FIG. 25 to FIG. 30.

FIG. 25 is a cross-sectional view obtained by cutting ultrasound probe 111 at third electrode 11-1 along the second direction (short-axis direction) in a case where switching was performed to connect a signal line (not shown) and third electrode 11-1. In the state illustrated in FIG. 25, although third electrode 11-1 is electrically connected with intermediate layer 7-1 and intermediate layer 7-5, third electrode 11-1 cannot electrically connect with intermediate layer 7-2, intermediate layer 7-3 and intermediate layer 7-4 due to the presence of insulating layer 19-12, insulating layer 19-13 and insulating layer 19-14. Consequently, when a voltage is applied to third electrode 11-1, although piezoelectric body 3-1 and piezoelectric body 3-5 can be driven through intermediate layers 7-1 and 7-5 and second electrodes 4-1 and 4-5, piezoelectric body 3-2, piezoelectric body 3-3 and piezoelectric body 3-4 cannot be driven.

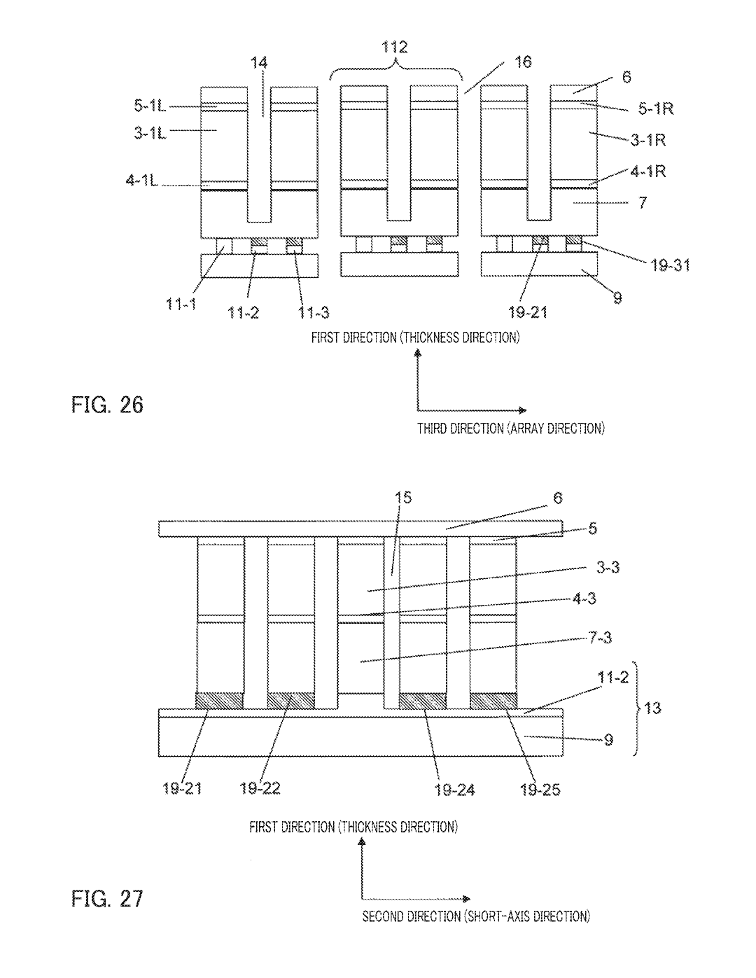

FIG. 26 is a cross-sectional view obtained by cutting ultrasound probe 111 at the position of piezoelectric body 3-1 along the third direction (array direction). One first groove 14 that penetrates through first electrode 5, piezoelectric body 3 and second electrode 4 and a part of intermediate layer 7 is provided in laminated body 112, and consequently piezoelectric body 3 is divided in two in the third direction (array direction). In the configuration shown in FIG. 26, first electrodes 5, piezoelectric bodies 3, and second electrodes 4 on the left and right sides obtained as a result of dividing piezoelectric body 3 in two are referred to as first electrodes 5-1L and 5-1R, piezoelectric bodies 3-1L and 3-1R, and second electrodes 4-1L and 4-1R, respectively. An electrical signal that passed through third electrode 11-1 is electrically connected with intermediate layer 7-1, and furthermore flows to piezoelectric body 3-1L and piezoelectric body 3-1R through second electrode 4-1L and second electrode 4-1R. In the present embodiment, even though first groove 14 penetrates through second electrode 4, because intermediate layer 7 through which first groove 14 does not penetrate completely is interposed between second electrode 4 and third electrode 11, a voltage can be applied to both piezoelectric body 3-1L and piezoelectric body 3-1R. The same also applies in the case of a cross section (not shown) obtained by cutting ultrasound probe 111 at the position of piezoelectric body 3-5, and is not limited to piezoelectric body 3-1.

FIG. 27 is a cross-sectional view obtained by cutting ultrasound probe 111 at third electrode 11-2 along the second direction (short-axis direction) in a case where switching was performed to connect a signal line and third electrode 11-2. In the state illustrated in FIG. 27, although third electrode 11-2 is electrically connected with intermediate layer 7-3, third electrode 11-2 cannot electrically connect with intermediate layer 7-1, intermediate layer 7-2, intermediate layer 7-4 and intermediate layer 7-5 due to the presence of insulating layer 19-21, insulating layer 19-22, insulating layer 19-24 and insulating layer 19-25. Consequently, when a voltage is applied to third electrode 11-2, although piezoelectric body 3-3 can be driven through intermediate layer 7-3 and second electrode 4-3, piezoelectric body 3-1, piezoelectric body 3-2, piezoelectric body 3-4 and piezoelectric body 3-5 cannot be driven.

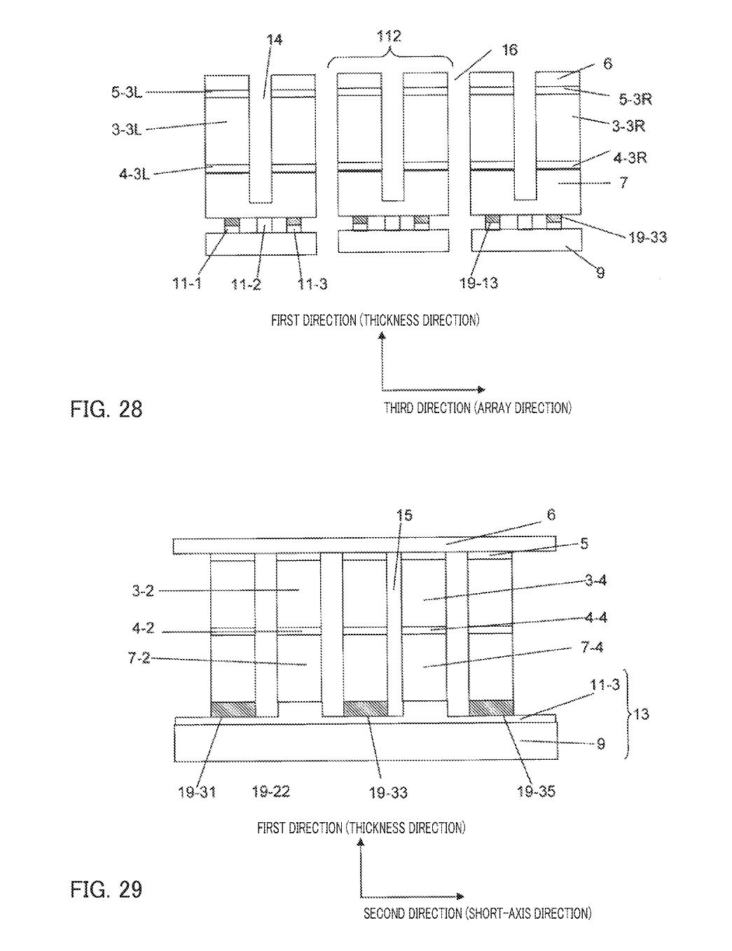

FIG. 28 is a cross-sectional view obtained by cutting ultrasound probe 111 at the position of piezoelectric body 3-3 along the third direction (array direction). One first groove 14 that penetrates through first electrode 5, piezoelectric body 3 and second electrode 4 and a part of intermediate layer 7 is provided in laminated body 112, and consequently piezoelectric body 3 is divided in two in the third direction (array direction). According to the configuration shown in FIG. 28, first electrodes 5, piezoelectric bodies 3, and second electrodes 4 on the left and right sides obtained as a result of dividing piezoelectric body 3 in two are referred to as first electrodes 5-3L and 5-3R, piezoelectric bodies 3-3L and 3-3R, and second electrodes 4-3L and 4-3R, respectively. An electrical signal that passed through third electrode 11-2 is electrically connected with intermediate layer 7-3, and furthermore flows to piezoelectric body 3-3L and piezoelectric body 3-3R through second electrode 4-3L and second electrode 4-3R. In the present embodiment, even though first groove 14 penetrates through second electrode 4, because intermediate layer 7 through which first groove 14 does not penetrate completely is interposed between second electrode 4 and third electrode 11, a voltage can be applied to both piezoelectric body 3-3L and piezoelectric body 3-3R.

FIG. 29 is a cross-sectional view obtained by cutting ultrasound probe 111 at third electrode 11-3 along the second direction (short-axis direction) in a case where switching was performed to connect a signal line (not shown) and third electrode 11-3. In the state illustrated in FIG. 29, although third electrode 11-3 is electrically connected with intermediate layer 7-2 and intermediate layer 7-4, third electrode 11-3 cannot electrically connect with intermediate layer 7-1, intermediate layer 7-3 and intermediate layer 7-5 due to the presence of insulating layer 19-31, insulating layer 19-33 and insulating layer 19-35. Consequently, when a voltage is applied to third electrode 11-3, although piezoelectric body 3-2 and piezoelectric body 3-4 can be driven through intermediate layers 7-2 and 7-4 and second electrodes 4-2 and 4-4, piezoelectric body 3-1, piezoelectric body 3-3 and piezoelectric body 3-5 cannot be driven.

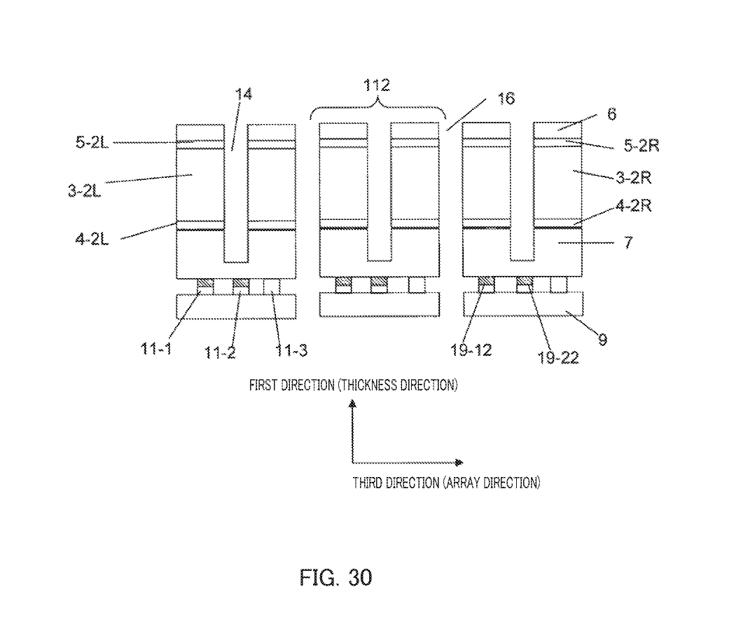

FIG. 30 is a cross-sectional view obtained by cutting ultrasound probe 111 at the position of piezoelectric body 3-2 along the third direction (array direction). One first groove 14 that penetrates through first electrode 5, piezoelectric body 3 and second electrode 4 and a part of intermediate layer 7 is provided in laminated body 112, and consequently piezoelectric body 3 is divided in two in the third direction (array direction). According to the configuration shown in FIG. 30, first electrodes 5, piezoelectric bodies 3, and second electrodes 4 on the left and right sides obtained as a result of dividing piezoelectric body 3 in two are referred to as first electrodes 5-2L and 5-2R, piezoelectric bodies 3-2L and 3-2R, and second electrodes 4-2L and 4-2R, respectively. An electrical signal that passed through third electrode 11-3 is electrically connected with intermediate layer 7-2, and furthermore flows to piezoelectric body 3-2L and piezoelectric body 3-2R through second electrode 4-2L and second electrode 4-2R. In the present embodiment, even though first groove 14 penetrates through second electrode 4, because intermediate layer 7 through which first groove 14 does not penetrate completely is interposed between second electrode 4 and third electrode 11, a voltage can be applied to both piezoelectric body 3-2L and piezoelectric body 3-2R. The same also applies in the case of a cross section (not shown) obtained by cutting ultrasound probe 111 at the position of piezoelectric body 3-4, and is not limited to piezoelectric body 3-2.

### Embodiment 3

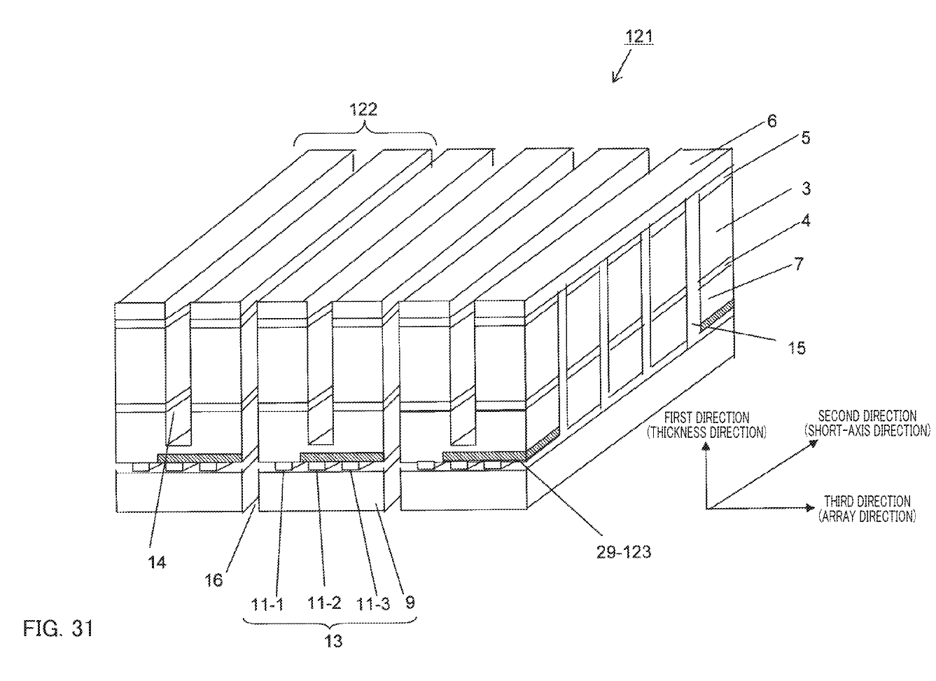

FIG. 31 is a perspective view illustrating an ultrasound probe according to Embodiment 3 of the present invention. While insulating layers 19 are provided on parts of third electrodes 11 in ultrasound probe 111 of Embodiment 2, according to the present embodiment insulating layers 29 are provided on intermediate layers 7 in the second direction (short-axis direction) as shown in FIG. 31. Other than this difference, the present embodiment is the same as Embodiment 2, and components in FIG. 31 that are common with FIG. 23 are denoted by the same reference numerals as in FIG. 23.

In a case where intermediate layer 7 is formed from a metallic material such as aluminum, insulating layer 29 is formed by selective anodizing. Further, in a case where intermediate layer 7 is formed from an insulating material, as shown in FIG. 6 and FIG. 7, by selectively forming the conductive layers, intermediate layer 7 at a portion at which a conductive layer is not provided may be used as insulating layer 29.

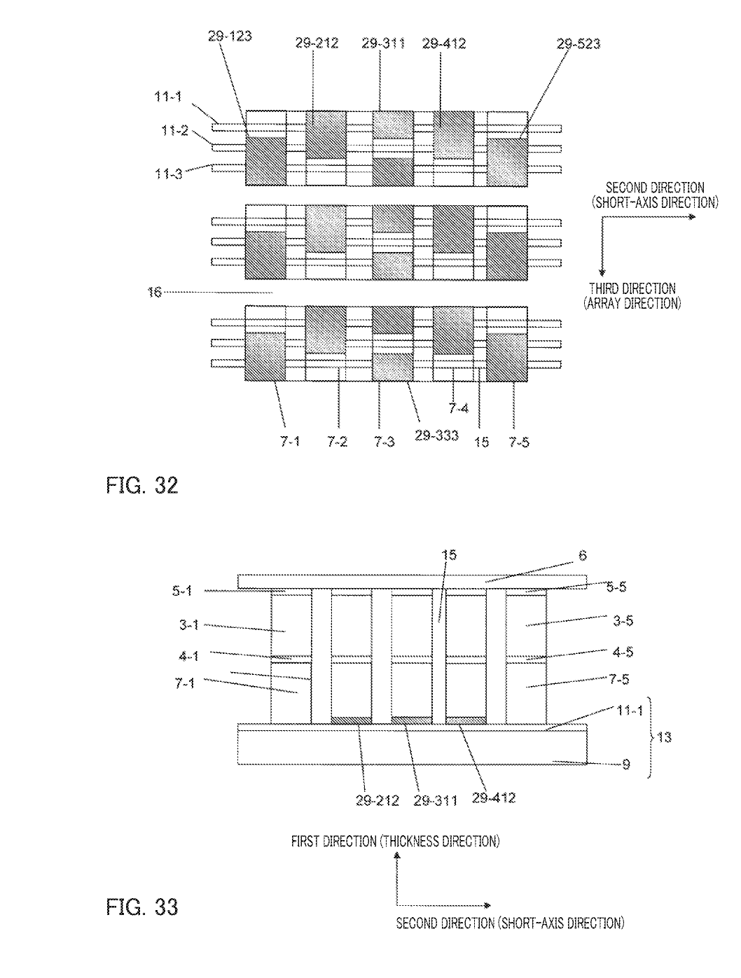

FIG. 32 illustrates an example of the arrangement of insulating layers 29. Insulating layer 29-123 shown in FIG. 32 is formed on the surface of intermediate layer 7-1 at a position facing third electrode 11-2 and third electrode 11-3. Insulating layer 29-212 is formed on the surface of intermediate layer 7-2 at a position facing third electrode 11-1 and third electrode 11-2. Insulating layer 29-311 is formed on the surface of intermediate layer 7-3 at a position facing third electrode 11-1. Insulating layer 29-333 is formed on the surface of intermediate layer 7-3 at a position facing third electrode 11-3. Insulating layer 29-412 is formed on the surface of intermediate layer 7-4 at a position facing third electrode 11-1 and third electrode 11-2. Insulating layer 29-523 is formed on the surface of intermediate layer 7-5 at a position facing third electrode 11-2 and third electrode 11-3.

(Operations of Ultrasound Probe 121 of Embodiment 3)

Operations of ultrasound probe 121 of Embodiment 3 will now be described using FIG. 33 to FIG. 38.

FIG. 33 is a cross-sectional view obtained by cutting ultrasound probe 121 at third electrode 11-1 along the second direction (short-axis direction) in a case where switching was performed to connect a signal line (not shown) and third electrode 11-1. In the state illustrated in FIG. 33, although third electrode 11-1 is electrically connected with intermediate layer 7-1 and intermediate layer 7-5, third electrode 11-1 cannot electrically connect with intermediate layer 7-2, intermediate layer 7-3 and intermediate layer 7-4 due to the presence of insulating layer 29-212, insulating layer 29-311 and insulating layer 29-412. Consequently, when a voltage is applied to third electrode 11-1, although piezoelectric body 3-1 and piezoelectric body 3-5 can be driven through intermediate layers 7-1 and 7-5 and second electrodes 4-1 and 4-5, piezoelectric body 3-2, piezoelectric body 3-3 and piezoelectric body 3-4 cannot be driven.

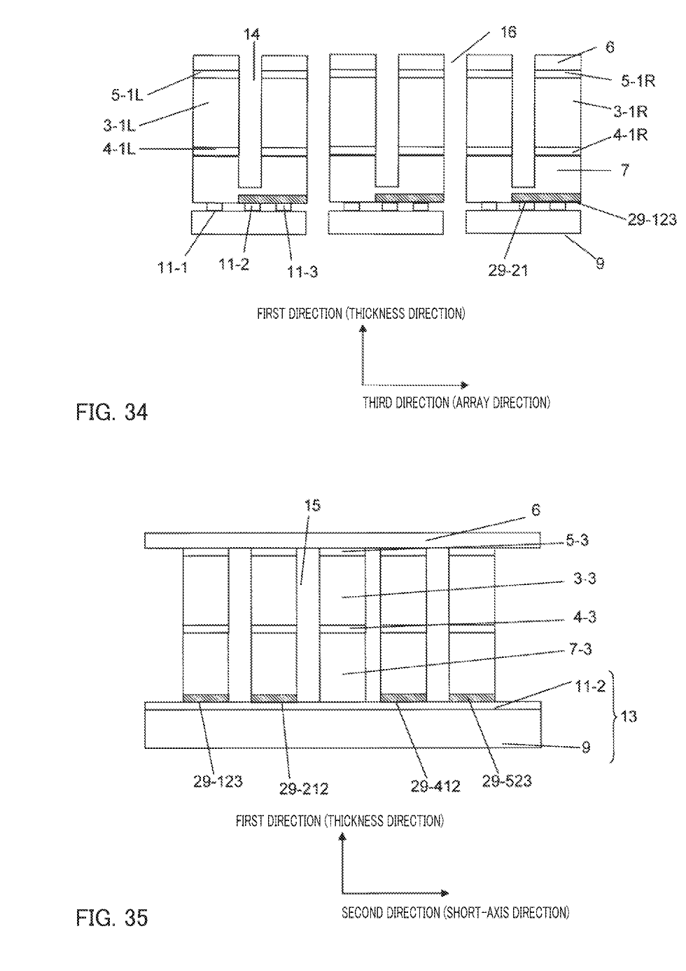

FIG. 34 is a cross-sectional view obtained by cutting ultrasound probe 121 at the position of piezoelectric body 3-1 along the third direction (array direction). One first groove 14 that penetrates through first electrode 5, piezoelectric body 3 and second electrode 4 and a part of intermediate layer 7 is provided in laminated body 122, and consequently piezoelectric body 3 is divided in two in the third direction (array direction). According to the configuration shown in FIG. 34, first electrodes 5, piezoelectric bodies 3, and second electrodes 4 on the left and right sides obtained as a result of dividing piezoelectric body 3 in two are referred to as first electrodes 5-1L and 5-1R, piezoelectric bodies 3-1L and 3-1R, and second electrodes 4-1L and 4-1R, respectively. An electrical signal that passed through third electrode 11-1 is electrically connected with intermediate layer 7-1, and furthermore flows to piezoelectric body 3-1L and piezoelectric body 3-1R through second electrode 4-1L and second electrode 4-1R. In the present embodiment, even though first groove 14 penetrates through second electrode 4, because intermediate layer 7 through which first groove 14 does not penetrate completely (does not penetrate through insulating layer 29 provided on the surface of intermediate layer 7) is interposed between second electrode 4 and third electrode 11, a voltage can be applied to both piezoelectric body 3-1L and piezoelectric body 3-1R. The same also applies in the case of a cross section (not shown) obtained by cutting ultrasound probe 121 at the position of piezoelectric body 3-5, and is not limited to piezoelectric body 3-1.

FIG. 35 is a cross-sectional view obtained by cutting ultrasound probe 121 at third electrode 11-2 along the second direction (short-axis direction) in a case where switching was performed to connect a signal line and third electrode 11-2. In the state illustrated in FIG. 35, although third electrode 11-2 is electrically connected with intermediate layer 7-3, third electrode 11-2 cannot electrically connect with intermediate layer 7-1, intermediate layer 7-2, intermediate layer 7-4 and intermediate layer 7-5 due to the presence of insulating layer 29-123, insulating layer 29-212, insulating layer 29-412 and insulating layer 29-523. Consequently, when a voltage is applied to third electrode 11-2, although piezoelectric body 3-3 can be driven through intermediate layer 7-3 and second electrode 4-3, piezoelectric body 3-1, piezoelectric body 3-2, piezoelectric body 3-4 and piezoelectric body 3-5 cannot be driven.

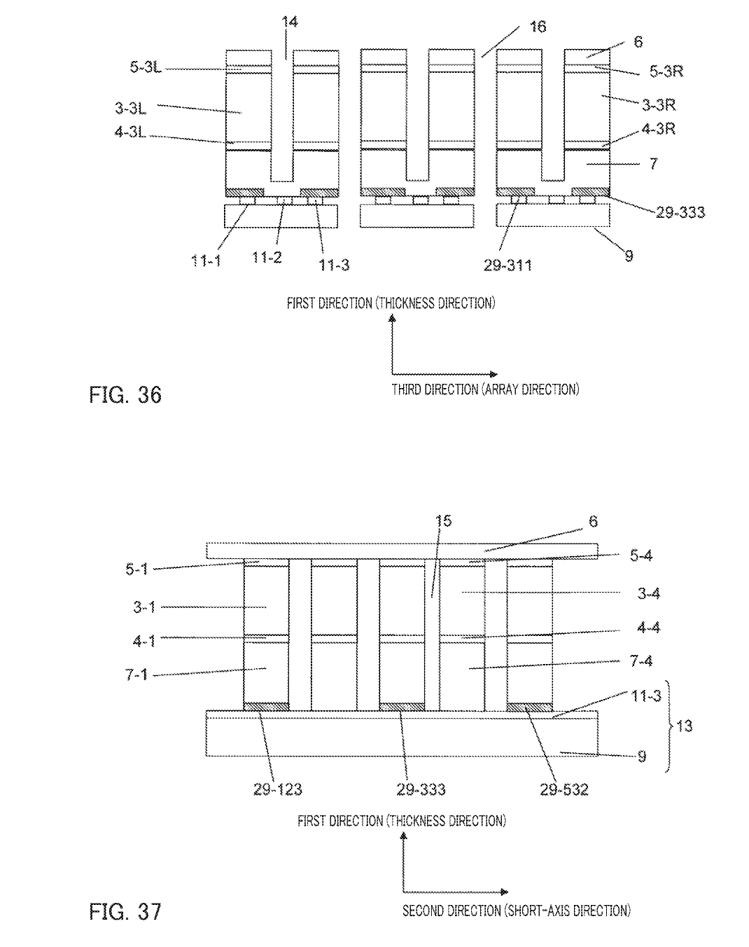

FIG. 36 is a cross-sectional view obtained by cutting ultrasound probe 121 at the position of piezoelectric body 3-3 along the third direction (array direction). One first groove 14 that penetrates through first electrode 5, piezoelectric body 3 and second electrode 4 and a part of intermediate layer 7 is provided in laminated body 122, and consequently piezoelectric body 3 is divided in two in the third direction (array direction). According to the configuration shown in FIG. 36, first electrodes 5, piezoelectric bodies 3, and second electrodes 4 on the left and right sides obtained as a result of dividing piezoelectric body 3 in two are referred to as first electrodes 5-3L and 5-3R, piezoelectric bodies 3-3L and 3-3R, and second electrodes 4-3L and 4-3R, respectively. An electrical signal that passed through third electrode 11-2 is electrically connected with intermediate layer 7-3, and furthermore flows to piezoelectric body 3-3L and piezoelectric body 3-3R through second electrode 4-3L and second electrode 4-3R. In the present embodiment, even though first groove 14 penetrates through second electrode 4, because intermediate layer 7 through which first groove 14 does not penetrate completely (does not penetrate through insulating layer 29 provided on the surface of intermediate layer 7) is interposed between second electrode 4 and third electrode 11, a voltage can be applied to both piezoelectric body 3-3L and piezoelectric body 3-3R.

FIG. 37 is a cross-sectional view obtained by cutting ultrasound probe 121 at third electrode 11-3 along the second direction (short-axis direction) in a case where switching was performed to connect a signal line (not shown) and third electrode 11-3. In the state illustrated in FIG. 37, although third electrode 11-3 is electrically connected with intermediate layer 7-2 and intermediate layer 7-4, third electrode 11-3 cannot electrically connect with intermediate layer 7-1, intermediate layer 7-3 and intermediate layer 7-5 due to the presence of insulating layer 29-123, insulating layer 29-333 and insulating layer 29-523. Consequently, when a voltage is applied to third electrode 11-3, although piezoelectric body 3-2 and piezoelectric body 3-4 can be driven through intermediate layers 7-2 and 7-4 and second electrodes 4-2 and 4-4, piezoelectric body 3-1, piezoelectric body 3-3 and piezoelectric body 3-5 cannot be driven.

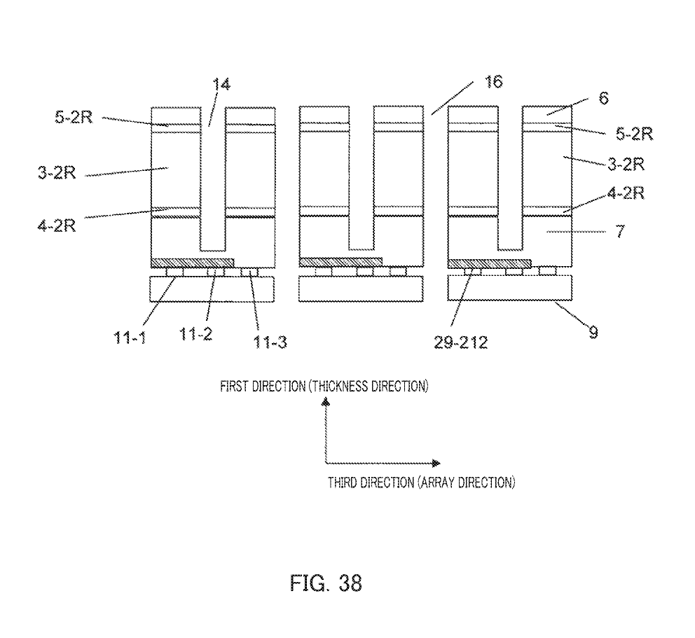

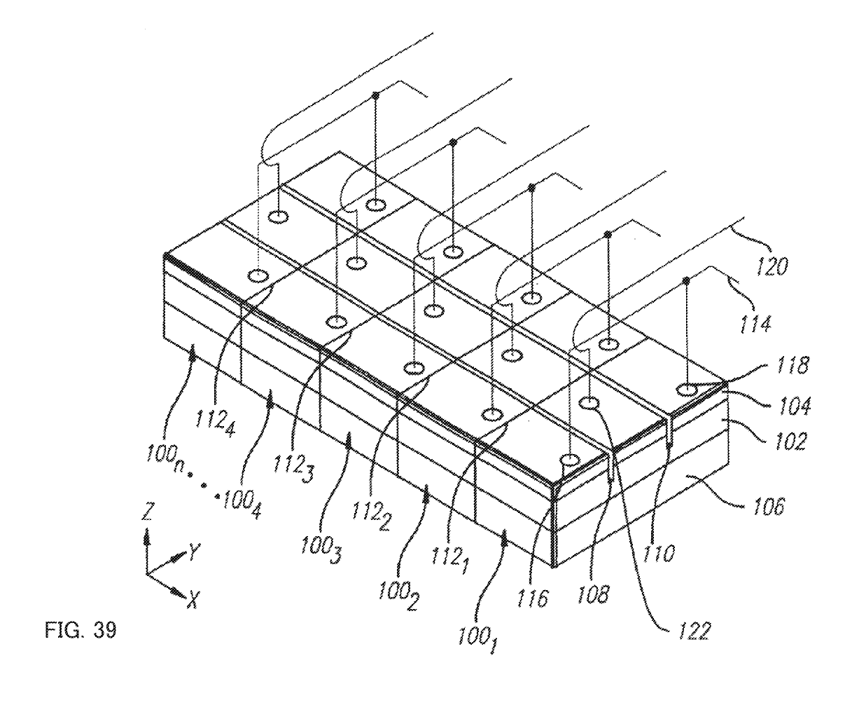

FIG. 38 is a cross-sectional view obtained by cutting ultrasound probe 121 at the position of piezoelectric body 3-2 along the third direction (array direction). One first groove 14 that penetrates through first electrode 5, piezoelectric body 3 and second electrode 4 and a part of intermediate layer 7 is provided in laminated body 122, and consequently piezoelectric body 3 is divided in two in the third direction (array direction). According to the configuration shown in FIG. 38, first electrodes 5, piezoelectric bodies 3, and second electrodes 4 on the left and right sides obtained as a result of dividing piezoelectric body 3 in two are referred to as first electrodes 5-2L and 5-2R, piezoelectric bodies 3-2L and 3-2R, and second electrodes 4-2L and 4-2R, respectively. An electrical signal that passed through third electrode 11-3 is electrically connected with intermediate layer 7-2, and furthermore flows to piezoelectric body 3-2L and piezoelectric body 3-2R through second electrode 4-2L and second electrode 4-2R. In the present embodiment, even though first groove 14 penetrates through second electrode 4, because intermediate layer 7 through which first groove 14 does not penetrate completely (does not penetrate through insulating layer 29 provided on the surface of intermediate layer 7) is interposed between second electrode 4 and third electrode 11, a voltage can be applied to both piezoelectric body 3-2L and piezoelectric body 3-2R. The same also applies in the case of a cross section (not shown) obtained by cutting ultrasound probe 121 at the position of piezoelectric body 3-4, and is not limited to piezoelectric body 3-2.

### INCORPORATION BY REFERENCE

The present application claims priority from Japanese application JP 2013-164537 filed on Aug. 7, 2013, the content of which is hereby incorporated by reference into this application.

### INDUSTRIAL APPLICABILITY

According to the ultrasound probe of the present invention, in an ultrasound probe which has a plurality of piezoelectric bodies arranged at predetermined intervals in a second direction (short-axis direction) and for which sub-dicing in the third direction (array direction) is required, an intermediate layer is provided between the plurality of piezoelectric bodies and a third electrode that controls the number of piezoelectric bodies to be driven (aperture). The ultrasound probe has a sub-diced configuration in which a first groove extending in the second direction penetrates as far as a part of the intermediate layer. Accordingly, electrical connections between the plurality of piezoelectric bodies and the third electrode can be realized with a high degree of reliability that is not affected by the finishing accuracy or component variations when manufacturing the ultrasound probe, and the ultrasound probe is useful as an ultrasound probe for ultrasound diagnostic imaging and the like.

### REFERENCE SIGNS LIST

1, 111, 121 Ultrasound probe
2, 112, 122 Laminated body
3 Piezoelectric body
4 Second electrode (signal electrode)
5 First electrode (ground electrode)
6 Ground layer
7 Intermediate layer
8 Double-sided FPC
9 Insulating layer
10 Fourth electrode
11 Third electrode
12 Conductive section
13 Single-sided FPC
14 First groove
15 Second groove
16 Third groove
19 Insulating layer
29 Insulating layer
31 Piezoelectric phase
32 Resin phase
33 Conductor layer
34 Insulator layer
35 Conductive layer
61 Transmitting circuit
62 Switching circuit
63 to 64 Switch
1101 to 1103 Signal line
1104 Ground line

What is claimed is:

1. An ultrasound probe, comprising a laminated body having:
a piezoelectric body having a predetermined thickness in a first direction;
a first electrode and a second electrode that face each other so as to sandwich the piezoelectric body in the first direction;
an intermediate layer that is electrically connected with the second electrode and is provided on an opposite side to the piezoelectric body with respect to the second electrode; and
a third electrode that faces the second electrode with the intermediate layer sandwiched therebetween, and extends in a second direction that is orthogonal to the first direction;
wherein:
a plurality of the first electrodes are arranged at predetermined intervals in the second direction and a plurality of the second electrodes are arranged at predetermined intervals in the second direction;
a plurality of the laminated bodies are arranged in a third direction that is orthogonal to the first direction and to the second direction; and
a first groove that penetrates through the first electrode, the piezoelectric body, the second electrode and a part of the intermediate layer and extends in the second direction is formed in the laminated body.

2. The ultrasound probe according to claim 1, wherein the laminated body further comprises:
a fourth electrode that is provided between the intermediate layer and the third electrode;
an insulating layer that is provided between the fourth electrode and the third electrode; and
a conductive section that penetrates through the insulating layer to electrically connect the fourth electrode with the third electrode.

3. The ultrasound probe according to claim 1, comprising a region in which an insulating layer is formed between a part of the third electrode and a part of the intermediate layer in the second direction.

4. The ultrasound probe according to claim 3, wherein a plurality of the third electrodes are formed in the third direction.

5. The ultrasound probe according to claim 1, wherein a second groove that penetrates through at least the first electrode, the piezoelectric body, the second electrode and the intermediate layer and that extends in the third direction is formed in the laminated body.

6. The ultrasound probe according to claim 1, wherein:
a plurality of the intermediate layers are arranged at predetermined intervals in the second direction, and
a distance between the intermediate layers that are adjacent in the second direction differs depending on a position thereof in the first direction.

7. The ultrasound probe according to claim 1, wherein:
the piezoelectric body is a composite structure in which a plurality of piezoelectric layers and resin layers are adjacent in the second direction; and
a width of the piezoelectric layer in the second direction is narrower than a width of the second electrode.

8. The ultrasound probe according to claim 1, wherein at least a part of a surface of the intermediate layer has electrical conductivity.

9. The ultrasound probe according to claim 1, wherein the intermediate layer is a conductor.

10. The ultrasound probe according to claim 1, wherein:
the intermediate layer is a composite structure in which a plurality of conductor layers and insulator layers are adjacent in the third direction, and
at least one of the conductor layers is arranged in regions facing the adjacent piezoelectric bodies with the first groove sandwiched therebetween in the third direction of the intermediate layer.

11. The ultrasound probe according to claim 1, wherein:
the intermediate layer is a laminated structure in which a plurality of conductor layers and insulator layers are arranged alternately side by side in the second direction, and
at least a part of the insulator layer is arranged in a region facing a region between the second electrodes that are adjacent in the second direction of the intermediate layer.

12. The ultrasound probe according to claim 1, wherein the intermediate layer has a portion that is formed across the plurality of the second electrodes.

13. The ultrasound probe according to claim 1, wherein an acoustic impedance of the intermediate layer is greater than or equal to an acoustic impedance of the piezoelectric body.

14. The ultrasound probe according to claim 1, wherein an acoustic impedance of the intermediate layer is less than an acoustic impedance of the piezoelectric body.

15. The ultrasound probe according to claim 1, wherein a thickness of the intermediate layer is equal to or greater than 0.01 mm.
